(19) United States
(12) Patent Application Publication
(10) Pub. No.: US 2024/0041645 A1
(43) Pub. Date: Feb. 8, 2024

(54) MEDICAMENT THERAPY DELIVERY SYSTEMS AND METHODS

(71) Applicant: W. L. Gore & Associates, Inc., Newark, DE (US)

(72) Inventors: Jérôme S. Conia, Phoenix, AZ (US); Jeffrey C. Towler, Wilmington, DE (US)

(21) Appl. No.: 18/230,907

(22) Filed: Aug. 7, 2023

Related U.S. Application Data
(60) Provisional application No. 63/395,501, filed on Aug. 5, 2022.

(51) Int. Cl. A61F 9/00

(52) U.S. Cl. CPC A61F 9/0017

(57) ABSTRACT

Medicament delivery devices are provided for metering delivery of a medicament over a time period to an ocular tissue. The device includes a tube, a microporous core, and a body portion with an external surface defining an internal reservoir. External surface defines a port of the body portion disposed to facilitate a first passage of the medicament from the reservoir. Tube length defines a second passage of the medicament from the first end to the second end of the tube. First end is coupled to the port to receive the first passage of the medicament from the reservoir. Second end is disposed at a distance from the first end to deliver the medicament to the ocular tissue. Microporous core is disposed in the lumen along at least a portion of the tube length and has a porosity that meters the second passage of the medicament.

### CROSS REFERENCE TO RELATED APPLICATION

This application claims the benefit of U.S. Provisional Application No. 63/395,501, filed Aug. 5, 2022, which is incorporated by reference herein in its entirety for all purposes.

### FIELD

The present disclosure relates generally to apparatuses and methods for delivering medicament into a tissue region of a body. More specifically, the disclosure relates to apparatuses and methods for delivering therapeutic medicament to the tissue pertaining to an eye.

### BACKGROUND

Aqueous humor is a fluid that fills the anterior chamber of the eye and contributes to the intraocular pressure or fluid pressure inside the eye. Ocular hypertension is a condition in the eye where the intraocular pressure or fluid pressure inside the eye is elevated. Untreated ocular hypertension can lead to disease, including glaucoma, which can result in a gradual and sometimes permanent loss of vision in the afflicted eye.

Many attempts have been made to treat ocular hypertension, and glaucoma in particular. Such attempts include surgical procedures that involve implantation of drainage devices designed to lower the intraocular pressure of the afflicted eye, as well as medicament administration. The goal of these treatments is to improve quality of life and to preserve visual function through a reduction of the intraocular pressure.

Though medicament administration is typically in the form of eyedrops that must be self-administered by the patient, implantable extended medicament delivery devices can be employed in certain instances. Implantable extended medicament delivery devices typically reside either on the exterior of the eye (e.g., extraocular approaches), or are alternatively implanted within the anterior chamber of the eye (intracameral approaches).

Extraocular approaches to medicament delivery present a variety of challenges. To be effective, the extraocular approach requires transporting, via the biological processes of the eye, a sufficient amount of the medicament through the conjunctival layer of the eye and into the anterior chamber of the eye. Obvious natural mechanisms such as the continual flushing mechanism of the human tear film, as well as the natural barrier to the interior of the eye formed by the conjunctiva, complicate the effectiveness of extraocular approaches, resulting in sub-optimal dose delivery over time. Extraocular approaches therefore sometimes include administration of an excessive amount of the medicament to extend a period of efficacy.

Intracameral approaches, on the other hand, are more invasive approaches requiring a puncture through the various tissue layers of the eye to gain access to, and placement of, the device within the anterior chamber of the eye. Intracameral approaches are additionally complicated where the medicament is administered in association with a device that is absorbable (bioabsorbable), as the degrading nature of the device may lead to the device dislodging and floating within the anterior chamber. Moreover, device removal and repeated replacement requires trauma to the tissues of the eye.

### SUMMARY

Disclosed herein are medicament delivery or metering devices and methods for metering delivery of a medicament over a time period to an ocular tissue. Advantages of such devices and methods including controlling the rate at which the medicament is delivered or distributed to a target location such as a target tissue for therapeutic treatment, as well as to facilitate transitioning of the medicament from its first state to its second state when it travels along a delivery passage to be delivered to the target location.

According to one example (“Example 1”), a medicament delivery device for metering delivery of a medicament over a time period to an ocular tissue is disclosed, where the device includes: a body portion having an external surface defining an internal reservoir, the external surface defining a port of the body portion disposed to facilitate a first passage of the medicament from the reservoir; a tube having a first end, a second end opposing the first end, and a lumen extending therebetween along a tube length, the tube length defining a second passage of the medicament from the first end to the second end, the first end coupled to the port to receive the first passage of the medicament from the reservoir, the second end disposed at a distance from the first end to deliver the medicament to the ocular tissue; and a microporous core disposed in the lumen along at least a portion of the tube length, the microporous core having a porosity that meters the second passage of the medicament.

According to another example (“Example 2”) further to Example 1, the medicament has a first state when disposed at the first end of the tube and a second state when disposed at the second end of the tube. The medicament transitions from the first state to the second state when travelling along the second passage through the microporous core.

According to another example (“Example 3”) further to Example 2, the first state being a delivery state of the medicament and the second state being a therapeutic state of the medicament.

According to another example (“Example 4”) further to Example 2, the first state being a non-therapeutic state of the medicament and the second state being a therapeutic state of the medicament.

According to another example (“Example 5”) further to Example 2, the first state being a high-concentration state of the medicament and the second state being a low-concentration state of the medicament. The low-concentration state has a lower concentration than the high-concentration state.

According to another example (“Example 6”) further to any one of Examples 1-5, the microporous core includes a plurality of nodes and fibrils configured to meter the second passage of the medicament.

According to another example (“Example 7”) further to Example 6, the second passage of the medicament is configured to be metered by a surface roughness and a surface energy of the plurality of nodes and fibrils of the microporous core.

According to another example (“Example 8”) further to Example 6 or 7, the microporous core comprises ePTFE fibers.

According to another example (“Example 9”) further to Example 8, the ePTFE fibers include a uniaxially expanded structure.

According to another example (“Example 10”) further to Example 8, the ePTFE fibers include a spiral or helicoidal geometry.

According to another example (“Example 11”) further to Example 6 or 7, the microporous core comprises ePTFE strips including a biaxially expanded structure.

According to another example (“Example 12”) further to any one of Examples 1-11, the tube comprises a thermoplastic compound.

According to another example (“Example 13”) further to Example 12, the thermoplastic compound comprises one or more of: FEP, EFEP, ETFE, or PATT.

According to another example (“Example 14”) further to any one of Examples 1-11, the tube comprises an elastomeric compound.

According to another example (“Example 15”) further to Example 14, the elastomeric compound comprises silicone.

According to another example (“Example 16”) further to any one of Examples 1-15, the tube is a coating disposed on an external surface of the microporous core.

According to another example (“Example 17”) further to any one of Examples 1-15, the tube is a tape wrapped around an external surface of the microporous core.

According to another example (“Example 18”) further to any one of Examples 1-17, the porosity of the microporous core is preselected to allow a passage of the medicament compound having a predetermined molecular shape.

According to another example (“Example 19”) further to any one of Examples 1-17, the porosity of the microporous core is preselected to allow a passage of the medicament compound having a predetermined molecular mass.

According to another example (“Example 20”) further to any one of Examples 1-17, the porosity of the microporous core is preselected to allow a passage of the medicament compound having a predetermined molecular polarity.

According to another example (“Example 21”) further to any one of Examples 1-20, the external surface of the body portion comprises a first surface and a second surface opposing the first surface, and the port is disposed between the first and second surfaces.

According to another example (“Example 22”) further to any one of Examples 1-20, the external surface of the body portion comprises a first surface and a second surface opposing the first surface, and the port is disposed in the first surface.

According to another example (“Example 23”) further to Example 22, the first end of the tube is disposed within the reservoir such that a majority of the tube length is disposed within the reservoir.

According to another example (“Example 24”) further to Example 23, the majority of the tube that is disposed within the reservoir is configured to partially cover the microporous core to provide a plurality of density diffusion openings.

According to another example (“Example 25”) further to Example 23 or 24, between 10% to 40% inclusively (or from 10% to 40%) of the tube length is located external to the reservoir.

According to another example (“Example 26”) further to any one of Examples 1-25, the body portion includes a resealable opening through which the reservoir is configured to be refilled with the medicament to be delivered.

According to one example (“Example 27”), a medicament metering device for metering delivery of a medicament over a time period to an ocular tissue is disclosed, where the medicament metering device includes: a tube having a first end, a second end opposing the first end, and a lumen extending therebetween along a tube length; and a microporous core disposed in the lumen along at least a portion of the tube length, the microporous core having a porosity that facilitates a metered passage of the medicament through the microporous core from the first end of the tube towards the second end of the tube. The medicament has a first state when disposed at the first end of the tube and a second state when disposed at the second end of the tube, the medicament transitioning from the first state to the second state when travelling along the metered passage. The first end of the tube includes a medicament-receiving portion sized to receive the medicament in the first state and positioned to channel the medicament in the first state to the microporous core.

According to another example (“Example 28”) further to Example 27, the first state being a delivery state of the medicament and the second state being a therapeutic state of the medicament.

According to another example (“Example 29”) further to Example 27, the first state being a non-therapeutic state of the medicament and the second state being a therapeutic state of the medicament.

According to another example (“Example 30”) further to Example 27, the first state being a high-concentration state of the medicament and the second state being a low-concentration state of the medicament, the low-concentration state having a lower concentration than the high-concentration state.

According to another example (“Example 31”) further to any one of Examples 27-30, the medicament-receiving portion comprises a microporous medicament-receiving component having a different porosity from the porosity of the microporous core.

According to another example (“Example 32”) further to any one of Examples 27-30, the first end is substantially closed and the medicament-receiving portion is a medicament-receiving space within the lumen in which the medicament is temporarily stored for the delivery.

According to another example (“Example 33”) further to any one of Examples 27-31, the first end is substantially open and the medicament-receiving portion is configured to receive the medicament from a surrounding environment external to the tube.

According to another example (“Example 34”) further to Example 33, the device further includes a filtering membrane disposed at the first end and configured to filter the medicament received from the surrounding environment.

According to another example (“Example 35”) further to Example 33, the first end is angulated with respect to the tube length.

According to another example (“Example 36”) further to Example 33 or 35, a section of the microporous core proximate to the first end of the tube is angulated with respect to the tube length.

According to another example (“Example 37”) further to any one of Examples 33-36, the device further includes a body portion having an external surface defining an internal reservoir. The external surface defines a port of the body portion coupled with the first end of the tube and disposed to deliver the medicament from the reservoir to the tube. The surrounding environment is the reservoir of the body portion.

According to another example (“Example 38”) further to any one of Examples 27-37, the microporous core includes a plurality of nodes and fibrils configured to control the metered passage of the medicament.

According to another example (“Example 39”) further to Example 38, the second passage of the medicament is configured to be metered by a surface roughness and a surface energy of the plurality of nodes and fibrils of the microporous core.

According to another example (“Example 40”) further to Example 38 or 39, the microporous core comprises ePTFE fibers.

According to another example (“Example 41”) further to Example 40, the ePTFE fibers include a uniaxially expanded structure.

According to another example (“Example 42”) further to Example 40, the ePTFE fibers include a spiral or helicoidal geometry.

According to another example (“Example 43”) further to Example 38 or 39, the microporous core comprises ePTFE strips including a biaxially expanded structure.

According to another example (“Example 44”) further to any one of Examples 27-43, the tube comprises a thermoplastic compound.

According to another example (“Example 45”) further to Example 44, the thermoplastic compound comprises one or more of: FEP, EFEP, ETFE, or PATT.

According to another example (“Example 46”) further to any one of Examples 27-43, the tube comprises an elastomeric compound.

According to another example (“Example 47”) further to Example 46, the elastomeric compound comprises silicone.

According to another example (“Example 48”) further to any one of Examples 27-47, the tube is a coating disposed on an external surface of the microporous core.

According to another example (“Example 49”) further to any one of Examples 27-47, the tube is a tape wrapped around an external surface of the microporous core.

According to another example (“Example 50”) further to any one of Examples 27-49, the porosity of the microporous core is preselected to allow a passage of the medicament compound having a predetermined molecular shape.

According to another example (“Example 51”) further to any one of Examples 27-49, the porosity of the microporous core is preselected to allow a passage of the medicament compound having a predetermined molecular mass

According to another example (“Example 52”) further to any one of Examples 27-49, the porosity of the microporous core is preselected to allow a passage of the medicament compound having a predetermined molecular polarity.

According to an example (“Example 53”), a method of metering delivery of a medicament to an ocular tissue is disclosed, where the method includes: disposing a medicament delivery device proximate to the ocular tissue, the medicament delivery device including an external surface defining an internal reservoir and a port disposed to facilitate a first passage of the medicament from the reservoir to the port; disposing a medicament delivery tube extending from the port at a first end of the tube to position a second end of the tube opposing the first end in a fluidic contact with the ocular tissue to facilitate a second passage of the medicament from the port to the second end of the tube; and delivering the medicament from the second end of the tube via a microporous core disposed in the tube, the microporous core having a porosity that meters the second passage of the medicament.

According to another example (“Example 54”), a method of metering delivery of a medicament to an ocular tissue is disclosed, where the method includes: disposing a first end of a medicament delivery tube proximate to the ocular tissue; disposing a second end of the medicament delivery tube opposing the first end of the medicament delivery tube to establish fluidic contact with the ocular tissue; and delivering the medicament from the second end of the tube via a microporous core disposed in the tube, the microporous core having a porosity that meters a passage of the medicament from the first end of the tube to the second end of the tube.

According to an example (“Example 55”) further to Example 53 or 54, the second end of the medicament delivery tube is disposed within an anterior chamber of an eye.

According to an example (“Example 56”) further to Example 53 or 54, the second end of the medicament delivery tube is disposed within a posterior chamber of an eye.

According to an example (“Example 57”) further to Example 53 or 54, the second end of the medicament delivery tube is disposed within a vitreous body of an eye.

According to an example (“Example 58”) further to Example 53 or 54, the second end of the medicament delivery tube is disposed between a sclera tissue and a choroid tissue of an eye.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the embodiments of the disclosure and are incorporated in and constitute a part of this specification, illustrate examples, and together with the description serve to explain the principles of the disclosure.

FIG. 1 is a line drawing of a medicament delivery device according to an embodiment disclosed herein;

FIGS. 2A through 2F are cross-sectional diagrams of an eye with the medicament delivery device implanted to facilitate medicament delivery to different locations therein according to embodiments disclosed herein;

FIG. 3A is a schematic diagram of the medicament delivery device as viewed from the top according to embodiments disclosed herein;

FIG. 3B is a cross-sectional view of the medicament delivery device of FIG. 3A as cut along the line B-B;

FIG. 3C is a cross-sectional view of the tube of the medicament delivery device of FIG. 3B as cut along the line C-C;

FIG. 3D is another cross-sectional view of the tube in FIG. 3C with its microstructure including nodes and fibrils shown for illustrative purposes;

FIGS. 4A through 4G are schematic cross-sectional diagrams of different medicament delivery tubes as used in the medicament delivery device according to embodiments disclosed herein;

FIGS. 5A through 5C are SEM images of a cross-sectional view of a microporous core as implemented in the medicament delivery device according to embodiments disclosed herein (the images are to the scales shown in the images);

FIG. 5D is an SEM image of the microporous core of FIG. 5A as viewed from an end along the direction of diffusion (the image is to the scale shown in the image);

FIGS. 6A through 6C are schematic diagrams of different medicament transport passages according to embodiments disclosed herein;

FIG. 7A is a line drawing of a medicament delivery device according to an embodiment disclosed herein;

FIGS. 7B through 7D are schematic cross-sectional diagrams of the medicament delivery device of FIG. 7A according to embodiments disclosed herein;

FIGS. 8A and 8B are schematic cross-sectional diagrams of the medicament delivery device as implanted according to embodiments disclosed herein;

FIGS. 9A and 9B are schematic cross-sectional diagrams of the medicament delivery device as implanted according to embodiments disclosed herein;

FIGS. 10A and 10B are schematic cross-sectional diagrams of the medicament delivery tube as implanted according to embodiments disclosed herein;

FIGS. 11A through 11D are schematic cross-sectional diagrams illustrating the different stages of medicament transport and delivery according to embodiments disclosed herein;

FIGS. 12A through 12D are schematic cross-sectional diagrams illustrating the different stages of medicament transport and delivery according to embodiments disclosed herein;

FIG. 13 is a schematic cross-sectional diagram illustrating medicament transport and delivery by diffusion via concentration difference according to embodiments disclosed herein; and

FIG. 14 is an SEM image of a cross-sectional view of a body portion of the medicament delivery device according to embodiments disclosed herein (the image is to the scale shown in the image).

It should be understood that the drawings and replicas of the photographs are not necessarily to scale, unless indicated otherwise. In certain instances, details that are not necessary for an understanding of the disclosure or that render other details difficult to perceive may have been omitted. It should be understood, of course, that the disclosure is not necessarily limited to the particular examples or embodiments illustrated or depicted herein.

### DETAILED DESCRIPTION

### Definitions and Terminology

This disclosure is not meant to be read in a restrictive manner. For example, the terminology used in the application should be read broadly in the context of the meaning those in the field would attribute such terminology. Persons skilled in the art will readily appreciate that the various embodiments of the inventive concepts provided in the present disclosure can be realized by any number of methods and apparatuses configured to perform the intended functions. It should also be noted that the accompanying figures referred to herein are not necessarily drawn to scale (unless indicated otherwise), but may be exaggerated to illustrate various aspects of the present disclosure, and in that regard, the figures should not be construed as limiting. Some figures do, however, represent anatomy and the positioning of embodiments relative to that anatomy and such representations should be understood to be scaled and positioned accurately, with some deviation permitted as the anatomical structures depicted will vary in size and position from person to person.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

The term “fibril” as used herein describes an elongated piece of material such as a polymer, where the length and width are substantially different from each other. For example, a fibril may resemble a piece of string or fiber, where the width (or thickness) is much shorter or smaller than the length. In some examples, a fibril may be smaller (microscopic) in width or thickness than a piece of fiber.

The term “node” as used herein describes a connection point of at least two fibrils, where the connection may be defined as a location where the two fibrils come into contact with each other, permanently or temporarily. In some examples, a node may also be used to describe a larger volume of polymer than a fibril and where a fibril originates or terminates with no clear continuation of the same fibril through the node. In some examples, a node has a greater width but a smaller length than the fibril.

As used herein, “nodes” and “fibrils” may be used to describe objects that are usually, but not necessarily, connected or interconnected, and have a microscopic size, for example. A “microscopic” object may be defined as an object with at least one dimension (width, length, or height) that is substantially small such that the object or the detail of the object is not visible to the naked eye or difficult, if not impossible, to observe without the aid of a microscope (including but not limited to a scanning electron microscope or SEM, for example) or any suitable type of magnification device.

### Description of Various Embodiments

The present disclosure relates to systems, devices, and methods for delivering a medicament to an eye of a patient. In various embodiments, the medicament is an ocular medicament that is configured to treat, for example, ocular hypertension and/or glaucoma, by causing the intraocular pressure to decrease from undesirably high levels that may lead to a gradual and sometimes permanent loss of vision in the afflicted eye. In various embodiments, medicament delivery devices according to the instant disclosure are configured to meter medicament release rates for one or more different medicaments, and thus may be configured to provide multiple different release rates, including multiple different release rates for multiple medicaments. Some examples of suitable ocular medicaments include therapeutic agents, such as prostaglandin analogs (PGAs) (e.g., latanoprost), or therapeutic agents from other medicament classes, including beta-blockers such as timolol, alpha-2-agonists such as brimonidine tartrate, or carbonic anhydrase inhibitors such as dorzolamide, compounds of carbonic anhydrase inhibitors and beta-blockers, and compounds of alpha-agonists and beta-blockers which may be administered in combination with PGAs.

In some embodiments, such medicament delivery devices are configured to be implanted and minimally invasively refillable one or more times in situ without requiring removal of the device from an implantation site. Given the size and subconjunctival target implantation locations, implantation procedures can be performed outside of the operating room, where needle puncture and small incisions are commonly performed. Additionally, some examples include features for helping reduce micro-movement between the devices and the tissue into which they are implanted. Micro-movement may be defined as small movements between the devices and the tissue, wherein the movements may be on a micrometer or millimeter, and microsecond or millisecond scale. Micro-movement sometimes leads to irritation of the surrounding tissue, which is known to lead to a foreign body tissue response that can cause excessive scar formation, eventual erosion of implanted devices, and/or site infection. In some examples, the target implantation locations may include subconjunctival and/or sub-Tenon locations of the eye.

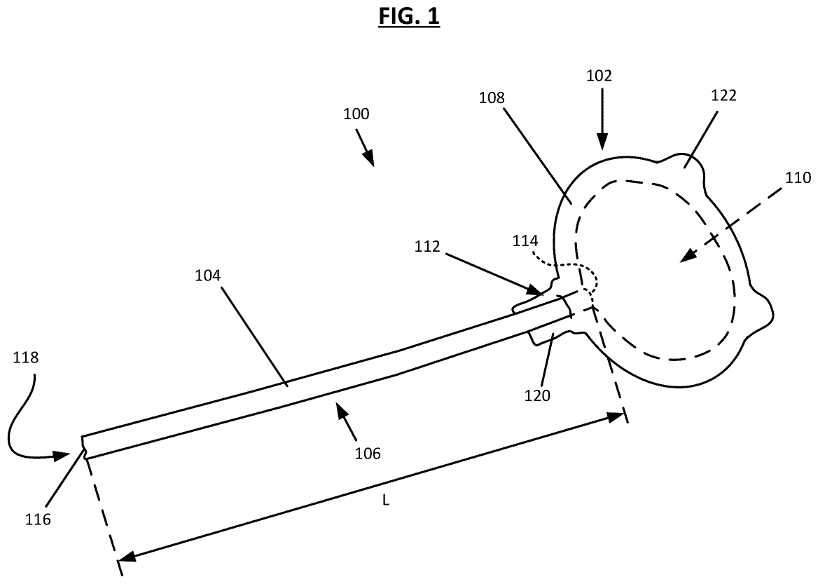

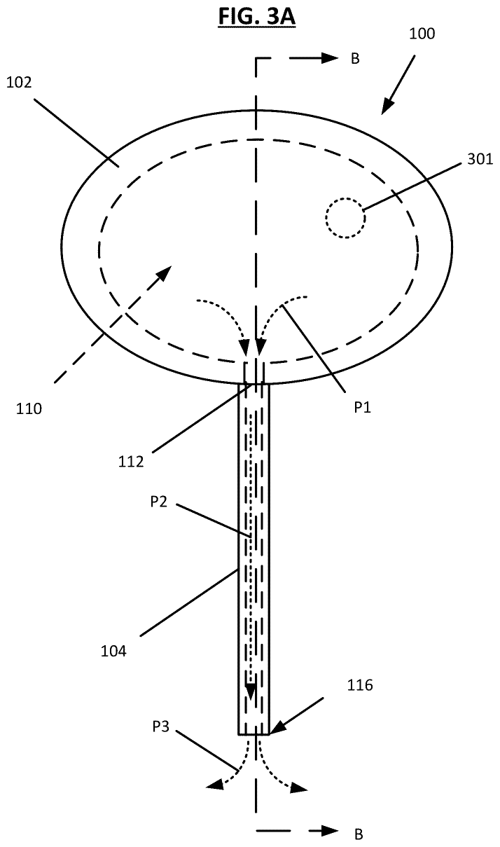

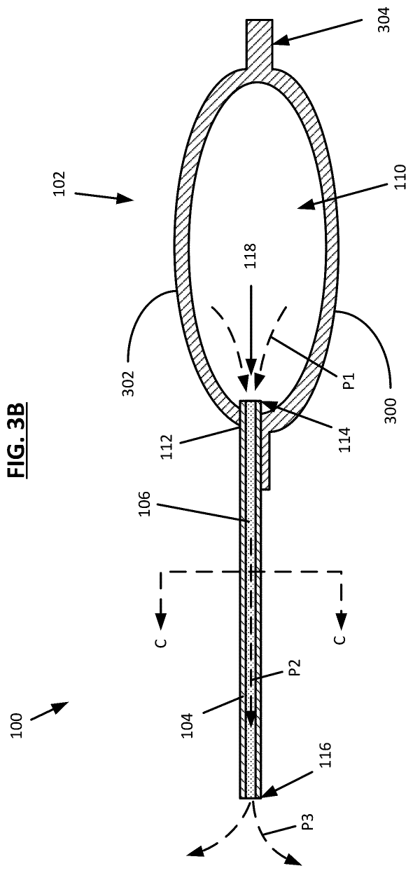

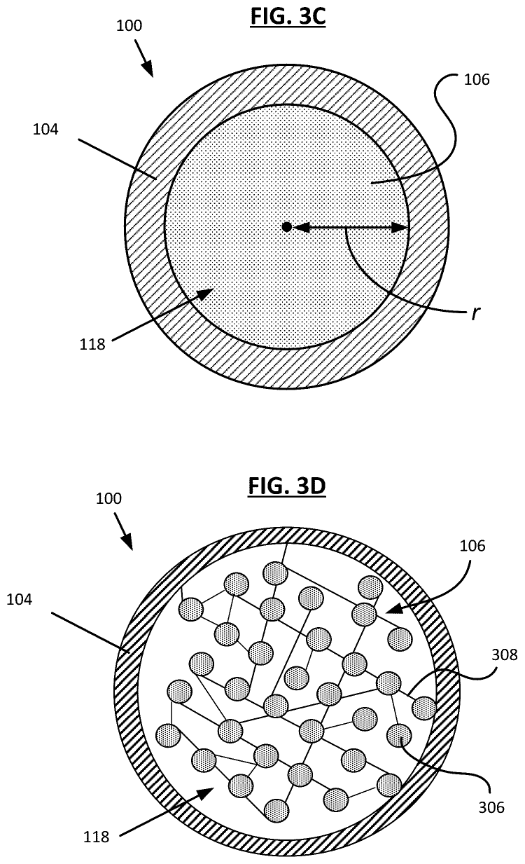

A medicament delivery device 100 for metering delivery of a medicament over a period of time to an ocular tissue according to some embodiments is illustrated in FIG. 1 (photograph) as well as FIGS. 3A and 3B (schematic). The device 100 includes a body portion 102, a tube 104, and a microporous core 106. The body portion 102 has an external surface 108 that defines an internal reservoir 110. The external surface 108 defines a port 112 of the body portion 102 that is disposed to facilitate a first passage “P1” of the medicament from the reservoir 110. The tube 104 has a first end 114, a second end 116 that opposes the first end 114, and a lumen 118 that extends between the two ends. The lumen 118 extends along a tube length “L” that is defined by the distance between the first end 114 and the second end 116.

In some examples, the body portion 102 may include a flap 120 to which the tube 104 may be attached or adhered, as well as a suture tab 122 which may be used to attach a suture to the body portion 102 without disturbing or affecting the reservoir 110.

The length L of the tube 104 defines a second passage “P2” of the medicament through the lumen 118, or more specifically through the microporous core 106 within the lumen 118. The second passage P2 extends from the first end 114 to the second end 116, and the medicament leaves the lumen 118 from the second end 116 via a third passage “P3” which allows the medicament to be delivered to the tissue of the target location. As such, the first end 114 is coupled to the port 112 to receive the first passage P1 of the medicament from the reservoir 110, and the second end 116 is disposed at a distance from the first end 114 (where the distance is the tube length L) to deliver the medicament to the target ocular tissue.

The microporous core 106 is disposed in the lumen 118 along at least a portion of the tube length L, and the core 106 has a porosity that meters the second passage P2 of the medicament as the medicament passes through the core 106. In some examples, the core 106 may extend the entire length L, while in other examples, the core 106 may extend a portion (e.g., less than about 95%, less than about 90%, less than about 85%, less than about 80%, less than about 75%, less than about 70%, less than about 65%, less than about 60%, less than about 55%, less than about 50%, or any other suitable range or value therebetween, according to the requirement of how much the medicament has to be metered as further explained herein) of the tube length L.

In some examples, the medicament may have a first state when disposed at the first end 114 of the tube 104, and may also have a second state when disposed at the second end 116 of the tube 104. The medicament may transition from the first state to the second state when traveling along the second passage P2 through the core 106.

In some examples, the first state is a delivery state of the medicament that enables the medicament to be delivered from the reservoir 110 to the core 106, and the second state is a therapeutic state of the medicament that enables the medicament to provide therapeutic treatment to the target tissue. In some examples, the first state is a non-therapeutic state of the medicament, and the second state is the therapeutic state. In some examples, the medicament may transition from the first state to the second state while within the reservoir 110 before moving through the first passage and traveling along the second passage through the core 106. In some examples, the first state is a high-concentration state (or an initial state) of the medicament, and the second state is a low-concentration state of the medicament (or a subsequent state of the medicament with a lower concentration than the initial state). The low-concentration state has a lower concentration than the high-concentration state.

In some examples, the external surface 108 of the device 100 may include a resealable opening 301 through which the medicament inside the reservoir 110 may be filled or refilled. In some examples, the external surface 108 of the device 100 may include a first surface 300 and a second surface 302 that opposes the first surface 300. In some examples, the port 112 may be disposed between the first surface 300 and the second surface 302 as shown in FIG. 3B. In some examples, the first surface 300 and the second surface 302 are two components or layers of material that is attached at a periphery 304 so as to form a single body portion 102 that defines the reservoir 110 therein. In some examples, the first surface 300 is the surface proximal to the eye or the surface that faces toward the eye, while the second surface 302 is the surface distal to the eye or the surface that faces away from the eye, after the device 100 is implanted.

In some embodiments, one or more of the first and second surfaces 300 and 302 may include a microporous microstructure. For example, one or more of the first and second surfaces 300 and 302 may include biocompatible materials such as expanded polytetrafluoroethylene (ePTFE). Additionally, one or more of first and second surfaces 300 and 302 may be formed of other biocompatible materials including biocompatible polymers, which may or may not be microporous, including, but not limited to, polyurethane, silicone, polysulfone, polyvinylidene fluorine (PVDF), polyhexafluoropropylene (PHFP), perfluoroalkoxy polymer (PFA), polyolefin, fluorinated ethylene propylene (FEP), acrylic copolymers, and polytetrafluoroethylene (PTFE).

The first and/or second surfaces 300 and 302 may be in the form of one or more sheets or films, and they may include knitted, woven, and/or non-woven forms including individual or multi-fiber strands. In some embodiments, the first and/or second surfaces 300 and 302 may be formed from a plurality of sheets or films of polymer material. In some embodiments, the sheets or films may be laminated or otherwise mechanically coupled together to form the first and/or second surfaces 300 and 302 as well as to form the body portion 102. Coupling of the sheets or films may be accomplished by a variety of mechanisms, including heat treatment, high pressure compression, bonding agents such as one or more adhesives, lamination, or other suitable methods known to one of skill in the art.

In some embodiments, adjacently-situated surfaces 300 and 302 and/or the layers of material forming such surfaces 300 and 302, may be partially or completely bonded via thermal methods where each of the polymers forming the materials are brought to or above their melting temperatures. In some embodiments, such thermal processes facilitate adhesive or cohesive bond formation between the materials or layers of material. In some embodiments, adjacently situated surfaces 300 and 302 and/or the layers of material forming such surfaces 300 and 302, may be partially bonded via thermal methods where at least one of the materials is brought to or above its melting temperature. Such thermal processes may facilitate adhesive or cohesive bond formation between the materials or layers of material. In some embodiments, one or more suitable adhesives are utilized and provide a sufficiently bonded interface. Adjacently situated surfaces 300 and 302 and/or the layers of material forming such surfaces 300 and 302 may be coupled together at one or more discrete locations (such as the periphery 304) to form stabilizing structures that extend through the resulting structure.

In some examples, the tube 104 and the core 106 may be formed of material or materials including but not limited to: PTFE, ePTFE, urethanes, polyurethane, silicones (organopolysiloxanes), polysulfone, PVDF, PHFP, PFA, polyolefin, FEP, ethylene fluorinated ethylene-propylene (EFEP), ethylene-tetrafluoroethylene (ETFE), 3′-(2-aminopyrimidyl)-2,2′:5′,2″-terthiophene (PATT), and acrylic copolymers, among others. In some embodiments, the materials may include other biocompatible polymers suitable for use in forming any one or more of the tube 104 and core 106 including, but not limited to, copolymers of silicon-urethane, styrene/isobutylene copolymers, polyisobutylene, polyethylene-co-poly(vinyl acetate), polyester copolymers, nylon copolymers, fluorinated hydrocarbon polymers and copolymers or mixtures of any of the foregoing may be used. In various embodiments, the elastomer or elastomeric material may include perfluoromethyl vinyl ether and tetrafluoroethylene, (per)fluoroalkylvinylethers (PAVE), a copolymer of tetrafluoroethylene and perfluoromethyl vinyl ether, silicone, a fluoroelastomer, a urethane, butyl rubber, styrene-butadiene, isobutylene-isoprene, or a TFE/PMVE copolymer.

In some examples, the core 106 is made of ePTFE fibers that are uniaxially or drawn or expanded to form a structure having the nodes and fibrils as disclosed herein. In some examples, the core 106 may be drawn or expanded in a spiral or helicoidal geometry to form such structure. In some examples, the core 106 may be made of ePTFE strips that are biaxially drawn or expanded to form a structure having the nodes and fibrils as disclosed herein. The strips may have a greater dimension (for example, greater width or thickness) than the fibers, for example. The tube 104 may be formed of an elastomeric compound including but not limited to silicone. In some examples, the elastomeric compound may be coated on an external surface of the core 106 to form a coating disposed on the external surface. In some examples, the elastomeric compound may be a tape that is wrapped around the external surface of the core 106 in order to form the tube 104 surrounding the core 106. Any other suitable methods of manufacturing or producing the tube 104 and the core 106 may be implemented as well.

In some examples, the device 100 may include one or more portions that are configured to promote or permit cellular infiltration and/or tissue attachment. The medicament may include a single therapeutic agent (e.g., a medication), or may include multiple therapeutic agents. The medicament may include additional materials (e.g. bioabsorbable polymers, pharmaceutically acceptable carrier) to affect the elution of the therapeutic agents (e.g. bioabsorbable polymers) from the delivery device. Throughout the description herein, the medicament may also be referred to as a drug or a pharmaceutical composition or combination as is may be composed of both a therapeutic agent and/or additional materials for effective elution of the therapeutic agent. For example, the medicament may include bioabsorbable microparticles having a size ranging between approximately 0.1 microns to 50 microns, or from approximately 1 micron to 50 microns, or from approximately 5 microns to 50 microns, or from approximately 15 microns to 50 microns, or from approximately 10 microns to 40 microns, or approximately from 15 microns to 25 microns, or approximately 18 microns to 23 microns. In some embodiments, the bioabsorbable microparticles have an average size of approximately 20 microns. In further embodiments, the therapeutic agent retained in the bioabsorbable microparticles may be latanoprost. The bioabsorbable microparticles may be retained within the device 100 during use, while the medicament may be released from the bioabsorbable microparticles, and as such, the device 100. The device 100 may be configured to meter medicament release rates for multiple different medicaments at multiple different release rates as explained herein.

As previously described, the medicament may be a pharmaceutical composition composed of at least one therapeutic agent and at least one additional material, such as, but not limited to, a bioabsorbable microparticle. The pharmaceutical composition is capable of adopting a first state and adopting a second state and is capable of transitioning between the first state and second state. For example, the pharmaceutical composition may transition from the first state to the second state when exposed to a fluid. In certain embodiments, the presence of the fluid may initiate a transition of the composition from the first state to the second state. In some embodiments, the first state corresponds to a non-therapeutic state and the second state correspond to a therapeutic state, such that in the absence of a sufficient amount of fluid, the therapeutic agent may not be released from the composition (and hence not administered to the patient in need thereof). In the therapeutic state, an amount of therapeutic agent released from the additional material may be increased relative to the amount released in the non-therapeutic state. In the therapeutic state, the therapeutic agent may be released in a pharmaceutical effective amount sufficient to treat the patient in need thereof. In some embodiments, the first state is substantially free of fluid.

In certain embodiments, the fluid comprises water. However, in other embodiments, various other fluids may also be present. In some embodiments, the time required for a provided composition to convert into a second state may vary in an application-appropriate manner, and may be relatively short. For example, in some embodiments, the pharmaceutical composition converts to a second state in a period of time between 10 seconds and 1 week after exposure to fluid (e.g., 30 seconds to 6 days, 1 minute to 5 days, 1 minute to 4 days, 1 minute to 3 days, 1 minute to 2 days, or 1 minute to 1 day).

Similarly, the time for a provided composition to remain in a second state may vary in an application-appropriate manner, and may be relatively long. For example, in some embodiments, the pharmaceutical composition may remain in a second state for a period of time between 1 min and 1 year after exposure to fluid and any time frame encompassed therein, such as 1 hour, 1 day, 1 week, 1 month, 2 months, 3 months, 4 months, 5 months, 8 months, or 11 months. Put differently, the quotidian influx of the medicament into the eye of the patient from the reservoir is consistent and controllable over the time period. Without wishing to be bound by any particular theory, this may be accomplished by using pharmaceutically acceptable carrier microparticles of various size and geometric complexities, where the conversion of the pharmaceutical composition occurs at different time points over the few months period based on the pace of degradation of the additional bioabsorbable material

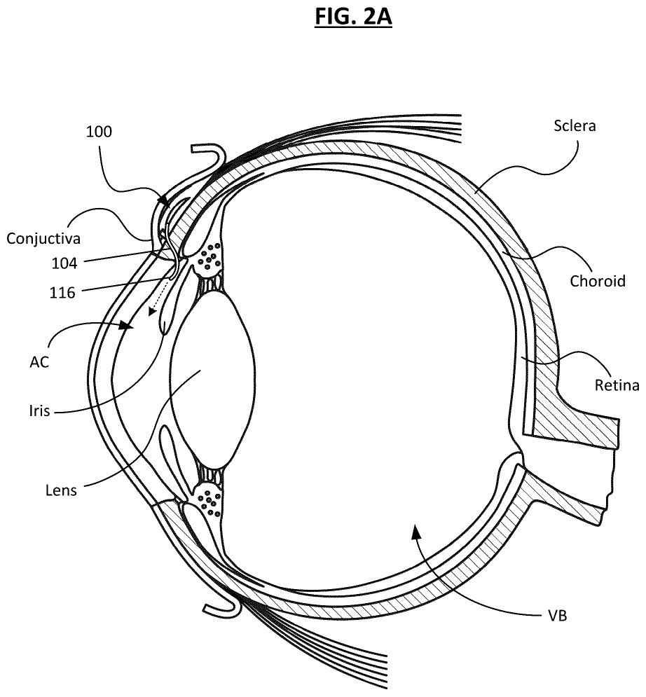

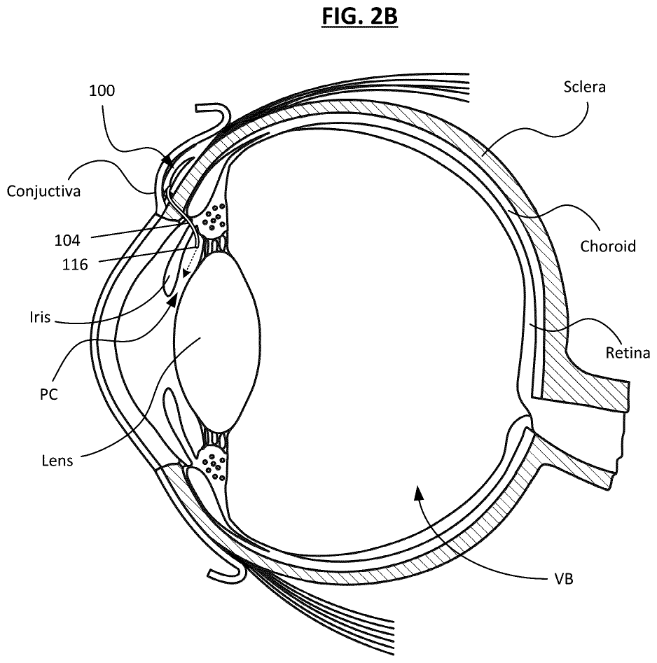

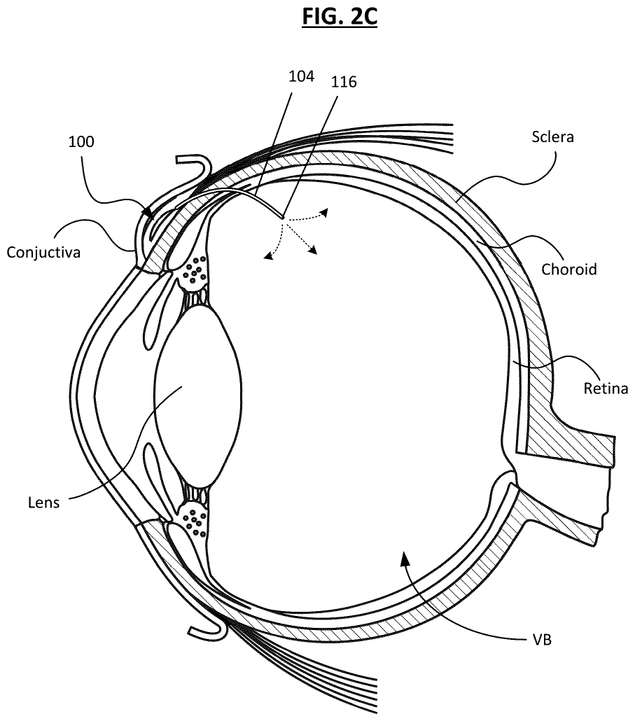

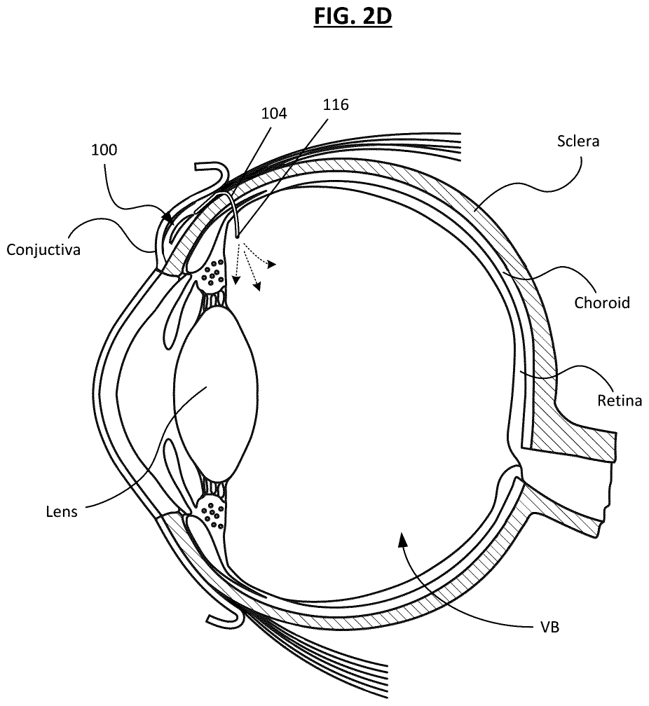

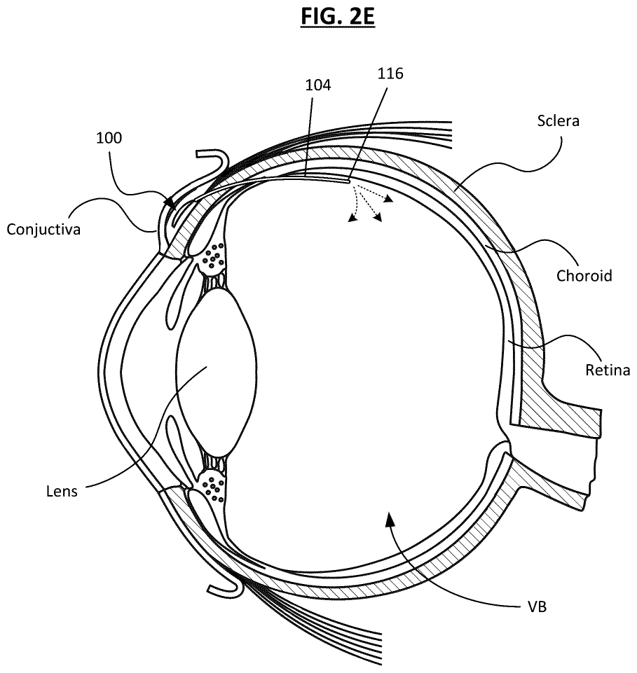

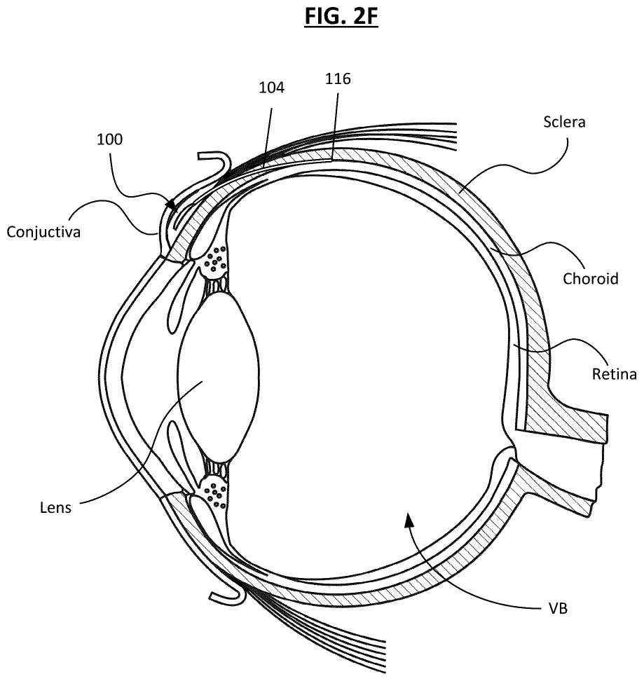

FIGS. 2A through 2F, illustrate some of the different examples of how the device 100 may be implanted within the eye, such as between the conjunctiva and the sclera of the eye. Also shown are an anterior chamber (AC), a posterior chamber (PC), a choroid, a retina, a lens, and a vitreous body (VB) as relevant to the implant.

In FIG. 2A, the tube 104 of the device 100 extends through the sclera into the AC, and the second end 116 is disposed inside the AC so the tube 104 is able to deliver medicament into the AC. In FIG. 2B, the tube 104 of the device 100 extends through the sclera into the PC, and the second end 116 is disposed inside the PC so the tube 104 is able to deliver medicament into the PC.

In FIGS. 2C through 2E, the tube 104 of the device 100 extends through the sclera and the choroid into the VB, and the second end 116 is disposed inside the VB so the tube 104 is able to deliver medicament into the VB. In FIG. 2C, the tube 104 is slightly curved so as to direct the second end 116 of the tube 104 toward the center of the eye. In FIG. 2D, the tube 104 is more angled or bent than the tube 104 in FIG. 2C, and the second end 116 is directed toward the front portion of the eye, e.g. toward the lens. In FIG. 2E, the tube 104 is less angled or straighter than the tube 104 in FIG. 2C, and the second end 116 is directed toward the back of the eye, e.g. toward the retina.

In FIG. 2F, the tube 104 is disposed such that the second end 116 is positioned between the choroid tissue and the sclera tissue so as to be able to deliver the medicament specifically to the region or area therebetween.

In example implementations, the medicament delivery device 100 can be useful for treating glaucoma. Glaucoma is a progressive vision loss associated with high intraocular eye pressure. For treatment of glaucoma, the medicament delivery device 100 can be at least partially subconjunctivally implanted (e.g., at or around a location between the limbus to the pars plana of the eye). Amongst other advantages, for example the reduced abrasion of tissue and the refillable element of the medicament delivery system, the use of the medicament delivery system described herein for treatment of glaucoma may overcome a common issue of patient compliance when using previously established treatment methods. For example, the use of an implantable medical delivery system reduces the chance that a patient may forget to administer a daily medicament, for example, through the use of eye drops.

In some examples, macular degeneration of the retina can be treated using example implementations of the medicament delivery device 100. Wet macular degeneration is a chronic eye disorder that involves abnormal blood vessel growth under the macula, which is responsible for central vision of the eye. In previously established treatments for macular degeneration, a needle is inserted into the eye and breaches the blood aqueous barrier. One of the advantages of the currently presented embodiment, amongst other advantages, is the ability for this invasive technique to be eliminated and for the treatment to be delivered minimally invasively. For treatment of macular degeneration, the medicament delivery device 100 can be at least partially subconjunctivally implanted and at least partially suprachoroidally implanted (e.g., posterior of the pars plana of the eye).

Other retinal diseases such as macular edema of the retina can be treated using example implementations of the medicament delivery device 100. Macular edema is a chronic eye disorder that involves distorted vision by swelling of the macula, which is responsible for central vision of the eye. For treatment of macular edema, the medicament delivery device 100 can be at least partially subconjunctivally implanted and at least partially suprachoroidally implanted (e.g., posterior of the pars plana of the eye). The medicament delivery device 100 can be implanted and arranged at one or more implantation locations or sites to deliver medicament for treatment of macular edema.

Another retinal disease that can be treated using example implementations of the medicament delivery device 100 is retinitis. Retinitis is a disease of the eye that involves inflammation of the retina. For treatment of retinitis, the medicament delivery device 100 can be at least partially subconjunctivally implanted and at least partially suprachoroidally implanted (e.g., posterior of the pars plana of the eye). In examples, the medicament delivery device 100 can be implanted and arranged at one or more implantation locations or sites to deliver medicament for treatment of retinitis.

Yet another retinal disease that can be treated using example implementations of the medicament delivery device 100 is retinoblastoma, which is a form of eye cancer. For treatment of retinoblastoma, the medicament delivery device 100 can be at least partially subconjunctivally implanted and at least partially suprachoroidally implanted (e.g., posterior of the pars plana of the eye). In examples, the medicament delivery device 100 can be implanted and arranged at one or more implantation locations or sites to deliver medicament for treatment of retinoblastoma.

Retinal vein occlusions are retinal diseases that can be treated using example implementations of the medicament delivery device 100. Retinal vein occlusions, such as CRVO and BRVO, are blockages of one or more retinal veins. These blockages can lead to excess blood and fluid in the retina. For treatment of retinal vein occlusions, a medicament delivery system can be at least partially subconjunctivally implanted and at least partially suprachoroidally implanted (e.g., posterior of the pars plana of the eye). In examples, the medicament delivery device 100 can be implanted and arranged at one or more implantation locations or sites to deliver medicament for treatment of retinal vein occlusions.

The present disclosure includes devices and methods suitable for treatment of corneal diseases. Some such corneal diseased include keratitis and dry eye. In some examples, the reservoir 110 or the tube 104 can dispense medicament from opposing sides of the reservoir 110 or the tube 104 such that the medicament is released in a direction toward and into the eye as well as in a direction away from the eye and into the conjunctiva.

Keratitis can be treated using example implementations of the medicament delivery device 100. Keratitis is inflammation of the cornea. For treatment of keratitis, the medicament delivery device 100 can be subconjunctivally implanted (e.g., near the lim bus the of the eye).

Dry eye is another corneal disease that can be treated using example implementations of the medicament delivery device 100. Dry eye is inadequate tear film lubrication of the eye. For treatment of dry eye, the medicament delivery device 100 can be subconjunctivally implanted. In examples, the medicament delivery device 100 can be implanted and arranged at one or more implantation locations or sites to deliver medicament for treatment of dry eye.

Still yet another disease that can be treated using example implementations of the medicament delivery device 100 is presbyopia, which is a crystalline stiffening of the lens of the eye. For treatment of presbyopia, a medicament delivery system can be subconjunctivally implanted. In examples, the medicament delivery device 100 can be implanted and arranged at one or more implantation locations or sites to deliver medicament for treatment of presbyopia.

In some examples, the device 100 may be used when gene therapy is deployed to prevent glaucomatous neurodegeneration for the treatment of age-related macular degeneration, retinitis pigmentosa, geographic atrophy, diametric macular edema, and diabetic retinopathy. In some examples, gene therapy is achieved by the sustained administration of either viral transducing vectors, adeno-associated virus (AAV) vectors, polymer-based nanoparticles, liposomes, or compacted nucleic acid nanoparticles.

In view of the above, the medicament delivery device 100 can be implanted in any suitable location of the eye according to the disease that is to be treated using the medicament such that the medicament to be administered can be effectively metered to the appropriate ocular tissue. For example, the medicament delivery device 100 may be disposed proximate to the ocular tissue. The tube 104 which may be a medicament delivery tube may extend from the port 112 of the body portion 102 (which facilitates the first passage P1 of the medicament from the reservoir 110 to the port 112) at the first end 114 of the tube 104 to position the second end 116 of the tube 104 in a fluidic contact with the ocular tissue to facilitate the second passage P2 of the medicament from the port 112 to the second end 116 of the tube 104. The medicament is delivered from the second end 116 via the microporous core 106 disposed in the tube 104 having a porosity that meters the second passage P2 of the medicament.

In some examples, the tube 104 of the medicament delivery device 100 may be disposed proximate to the ocular tissue. The second end 116 of the tube 104 may be disposed to establish a fluidic contact with the ocular tissue. The medicament may be delivered from the second end 116 via the core 106 disposed in the tube 104 having a porosity that meters the passage P2 of the medicament form the first end 114 to the second end 116 of the tube 104.

As shown in FIGS. 3C and 3D, the core 106 may be disposed within the tube 104 such that the core 106 substantially fills the inside of the tube 104, or the lumen 118 of the tube 104, which has an inner radius “r” as shown. The core 106 may comprise a plurality of nodes 306 and fibrils 308 which may be interconnected to each other as well as to the surrounding tube 104. In different examples, the nodes 306 and fibrils 308 may be positioned and interconnected in different ways so as to control the flow of second passage P2 of the medicament in different ways, for example to allow faster flow or slower flow, as further explained herein. In some examples, the passage P2 of the medicament is controlled or metered by a surface roughness and a surface energy of the nodes 306 and fibrils 308 of the core 106. The surface roughness of the nodes 306 and fibrils 308 may cause frictional forces that control or limit the flow of medicament (fluid and particles) through the core 106. The surface energy of the nodes 306 and fibrils 308 may cause intermolecular bonds of the medicament to be disrupted, thus controlling or limiting the flow of the medicament through the core 106.

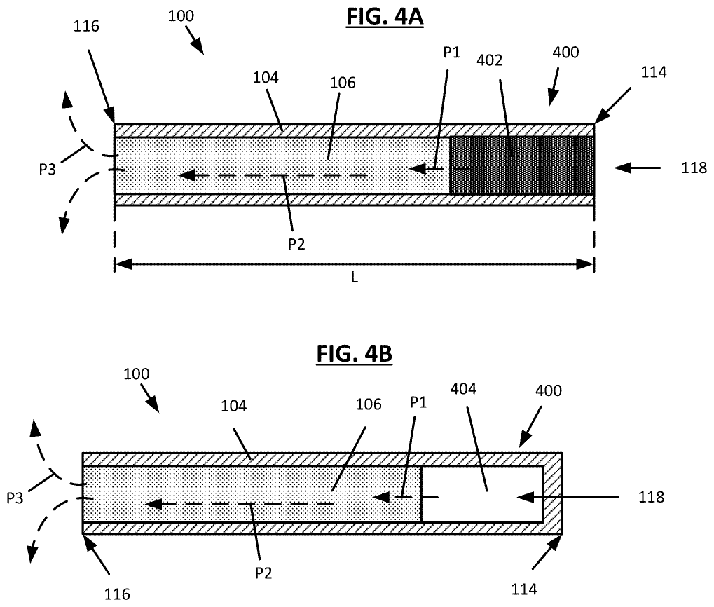

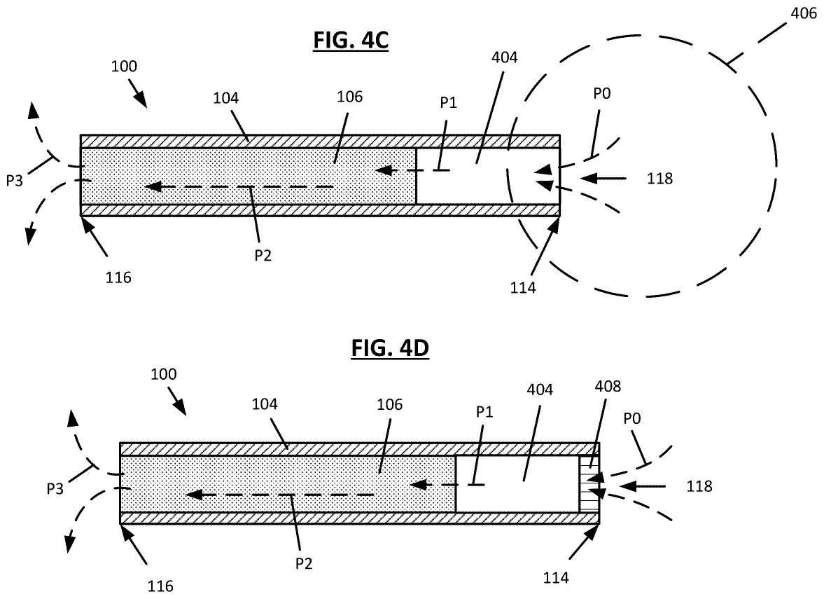

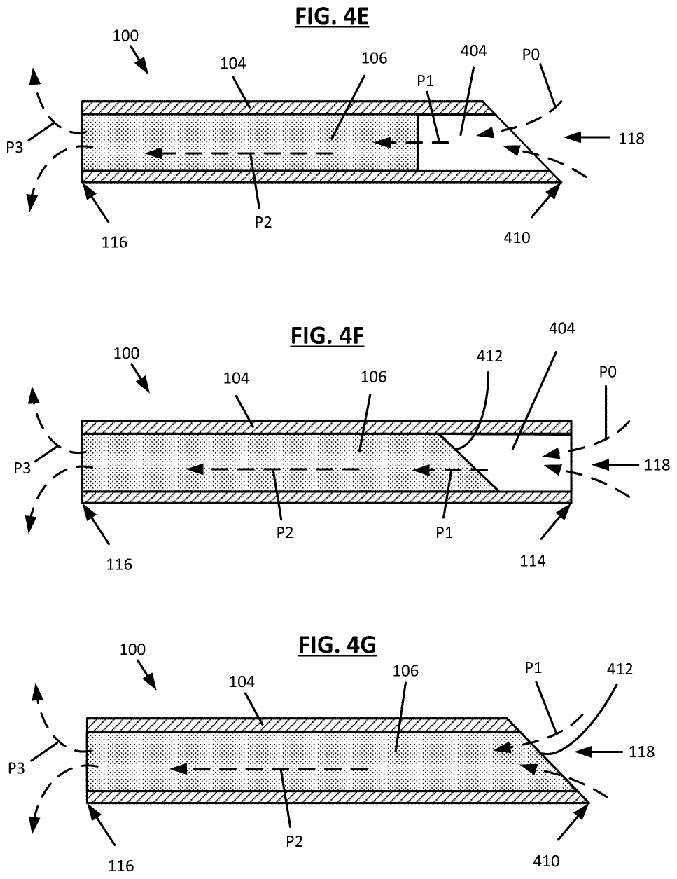

FIGS. 4A through 4G illustrate different examples of embodiments as disclosed herein where the medicament delivery device 100 includes the tube 104 and the microporous core 106 but may or may not include the body portion 102 as previously disclosed. In the absence of the body portion 102, the device may alternatively be referred to as a “medicament metering device” due to the tube 104 and the core 106 facilitating metering of the medicament that is to be transported therethrough. The tube 104 has the first end 114, the second end 116 opposing the first end 114, and the lumen 118 extending along the tube length L. The microporous core 106 is disposed in the lumen 118 along at least a portion (partially or entirely) of the tube length L. The core 106 has a porosity that facilitates a metered passage, shown as P2 in the figures, of the medicament through the core 106 from the first end 114 towards the second end 116. The medicament may have a first state when disposed at the first end 114 and a second state when disposed at the second end 116, transitioning from the first to second state when traveling along the metered passage (P2).

The first end 114 of the tube 104 includes a medicament-receiving portion 400 that is sized to receive the medicament in the first state. The medicament-receiving portion 400 is positioned to channel the medicament in the first state to the core 106. The medicament is channeled from the medicament-receiving portion 400 to the core 106 via the first passage P1 as shown.

In FIG. 4A, the medicament-receiving portion 400 includes a microporous medicament-receiving component 402 which has a different porosity from the porosity of the core 106. For example, the component 402 may be made of the same or different material with respect to the material of the core 106 and is structured or manufactured to gradually release the medicament stored in the component 402 into the core 106 via the first passage P1, after which the medicament travels through the core 106 via the second/metered passage P2, and eventually released from the second end 116 of the tube 104 via the third passage P3.

It is to be understood that the first end 114 may be substantially closed or substantially open. If substantially closed, the medicament stored in the component 402 is prevented from being released to the environment via the first end 114. If substantially open, the component 402 may be formed as a seal to prevent the medicament from being released via the substantially open first end 114. In some examples, such seal may be formed by either attaching or fusing the end of the component 402 that does not come into contact with the core 106 with another component that has zero or very low porosity as compared to the porosity of the component 402 (such that the very low porosity may be almost negligible as compared to the porosities of the core 106 and the component 402, for example). In some examples, the seal is formed by applying a certain treatment to the end of the component 402, such as melting, collapsing, tying, or any other suitable means that can be applied so that the porosity of the treated end of the component 402 (that does not come into direct contact with the core 106) may be sufficiently reduced so as to facilitate reduction of the medicament being released via the substantially open first end 114.

In FIG. 4B, the first end 114 is substantially closed, and the medicament-receiving portion 400 includes a medicament-receiving space 404 which is defined as a portion of the lumen 118 that is surrounded on one side by the closed first end 114 of the tube 104, and on the other side by the end portion of the core 106 that is proximal to the first end 114. The space 404 may initially or temporarily store the medicament inside such that the medicament gradually travels into the core 106 via the first passage P1, and the medicament then travels through the core 106 via the second/metered passage P2, before being released from the lumen 118 of the tube 104 via the third passage P3.

In FIGS. 4C through 4F, the first end 114 is substantially open and the medicament-receiving portion 400 (which may be shown in some of the figures to include the medicament-receiving space 404 but also may alternatively or additionally include the microporous medicament-receiving component 402, as suitable) receives the medicament from a surrounding environment 406 external to the tube 104. The environment 406 may be a different area of the body from the target area to which the medicament is to be delivered. For example, the environment 406 which initially and temporarily stores the medicament may be the subconjunctival space in which the first end 114 is disposed, and the second end 116 may be disposed in a different location such as in the AC (FIG. 2A), the PC (FIG. 2B), the VB (FIGS. 2C through 2E), or between the sclera and the choroid (FIG. 2F), among any other suitable locations. As such, an originating passage “P0” is defined in these examples showing a passage through which the medicament travels from the environment 406 into the medicament-receiving portion 400, before the medicament is passed into the core 106 from the medicament-receiving portion 400 via the first passage P1.

In FIG. 4C, the originating passage P0 and the first passage P1 may be considered as substantially the same passage, since the medicament does not change state or property from the passage P0 to the passage P1. In FIG. 4D, however, the medicament is filtered as it travels via the originating passage P0 through a filtering portion or membrane 408 disposed at the first end 114 of the tube 104. The portion or membrane 408 filters the medicament as it is received from the environment 406 such that undesired tissue or foreign particles may be filtered so as to reduce the amount of unwanted material that is allowed to enter the core 106.

In FIGS. 4E and 4F, one of the first end 114 or the core 106 may have an angulated section that is either cut or truncated at a substantially angled configuration with respect to the longitudinal axis of the tube 104. In FIG. 4E, the first end 114 is angulated to form an angulated first end 410. In FIG. 4F, the end portion of the core 106 that is proximal to the first end 114 is angulated to form an angulated section 412. The purpose of the angulation is to increase the cross-sectional area of the end portion that is angulated such that more medicament may be allowed to pass through as compared to before the angulation (that is, when the end portion is substantially perpendicular with respect to the longitudinal axis of the tube 104. Specifically, the angulated first end 410 facilitates increased flow of the passage P0, and the angulated section 412 of the core 106 facilitates increased flow of the passage P1.

In FIG. 4G, the first end 114 and the core 106 are both angulated such that the angulated first end 410 of the tube 104 and the angulated section 412 of the core 106 are flush with one another. This may be achieved by cutting or truncating both the first end 114 and the core 106 at an angle at the same time.

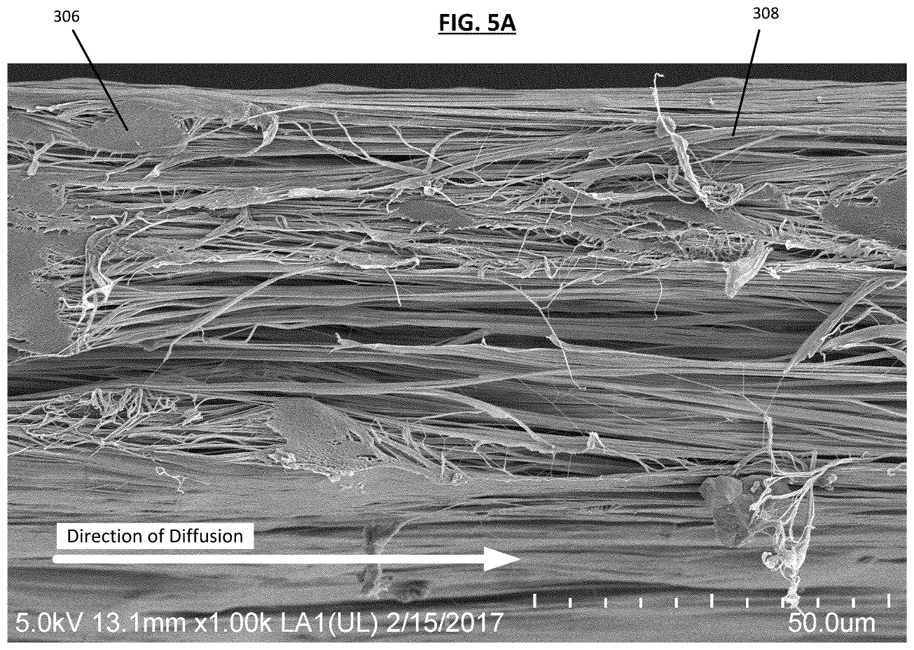

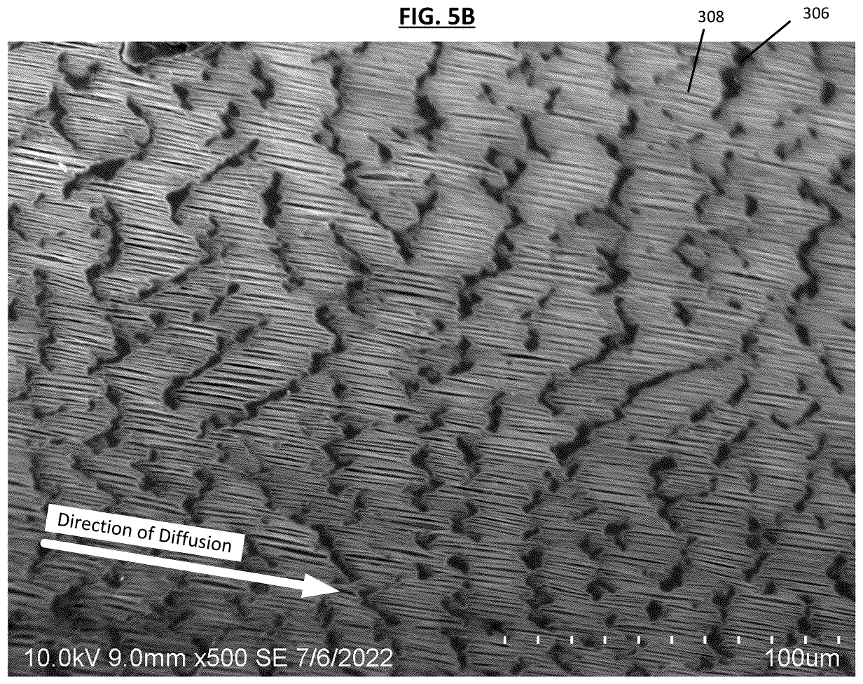

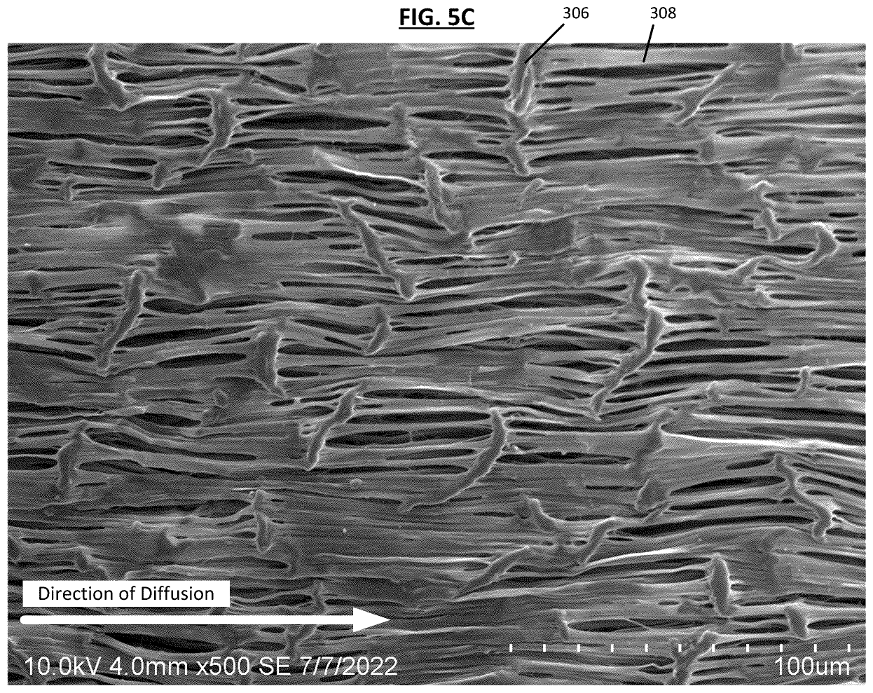

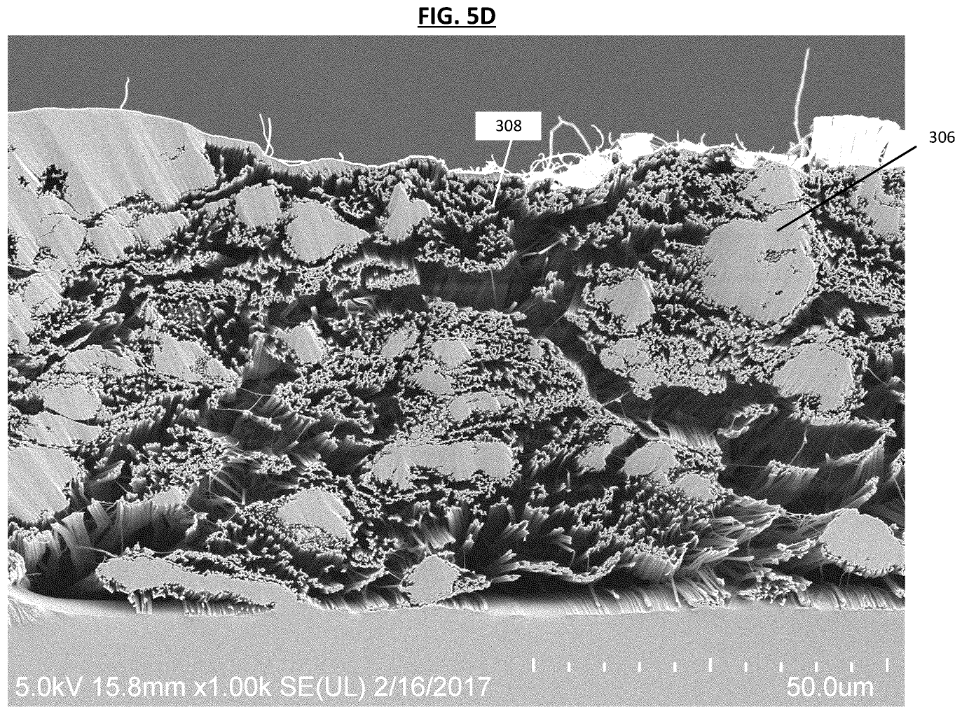

FIGS. 5A through 5D show different configurations of the nodes 306 and fibrils 308 forming the core 106 according to embodiments disclosed herein, where the direction of diffusion is indicated in FIGS. 5A through 5C. Displayed at the bottom of FIG. 5A is: “5.0 kV 13.1 mm×1.00 k LA1(UL) Feb. 15, 2017,” and the distance between two consecutive lines as shown at the bottom right hand corner represents 5.0 μm. Displayed at the bottom of FIG. 5B is: “10.0 kV 9.0 mm×500 SE Jul. 6, 2022,” and the distance between two consecutive lines as shown at the bottom right hand corner represents 10 μm. Displayed at the bottom of FIG. 5C is: “10.0 kV 4.0 mm×500 SE Jul. 7, 2022,” and the distance between two consecutive lines as shown at the bottom right hand corner represents 10 μm.

FIG. 5D shows the cross-sectional view of the core 106 similar to FIG. 3D. Displayed at the bottom of FIG. 5D is: “5.0 kV 15.8 mm×1.00 k SE(UL) Feb. 16, 2017,” and the distance between two consecutive lines as shown at the bottom right hand corner represents 5.0 μm. As shown, the nodes 306 may be formed by clumping or fusing together a plurality of fibrils 308 so as to reduce the rate of diffusion therethrough when the medicament travels through the core 106 in the direction of diffusion. In some examples, the direction of diffusion defines the general direction at which the medicament is configured to travel via the passage P2.

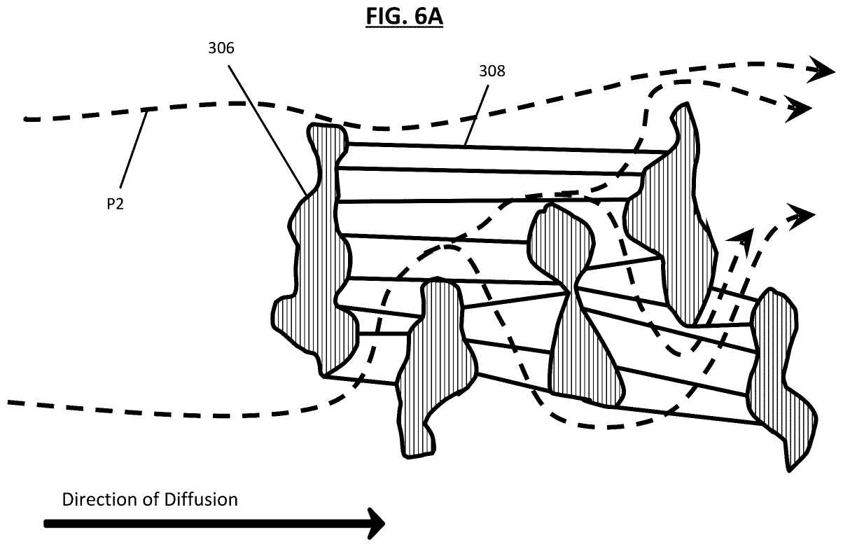

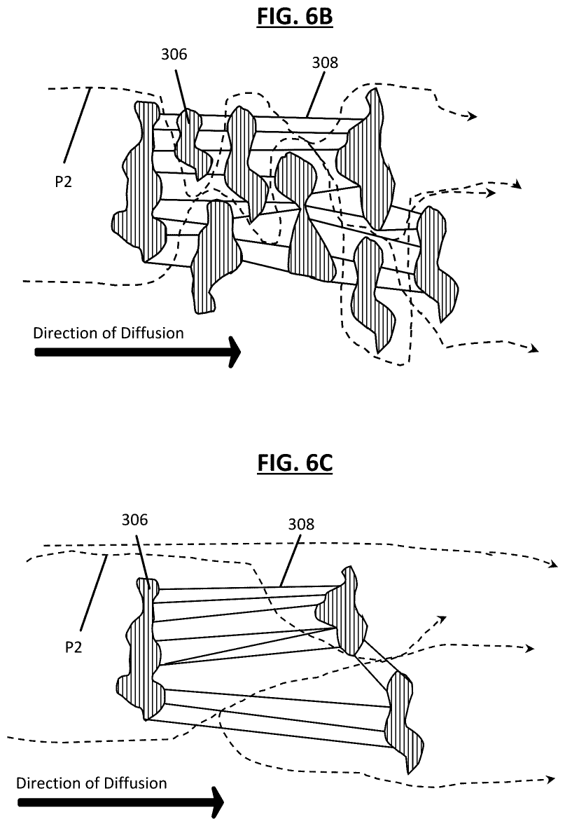

FIGS. 6A through 6C illustrate different examples of how the nodes 306 and fibrils 308 may meter the flow of medicament by affecting the rate of transport of the passage P2 taken by the medicament as it passes through the core 106 in the direction of diffusion as shown. The number of nodes 306 and fibrils 308 as well as their positioning with respect to each other may greatly affect the rate at which the medicament transport takes place (e.g., the rate at which the medicament travels from the first end 114 to the second end 116 of the tube 104).

According to Fick's First Law, the flux, or the amount of medicament traveled per unit time per unit area, can be expressed using the following equation:

$$J = - D \frac{dC}{dx} = \frac{{rate}_{drug}}{A} \qquad ( Equation 1 )$$

which can be written as the following equation:

$${rate}_{drug} = - D \frac{dC}{dx} * A \qquad ( Equation 2 )$$

where J is the flux, A is the inner area of the tube (i.e., the cross-sectional area of the lumen of the tube in which the core is disposed to fill), D is the diffusivity of the species (i.e., medicament), dC is the concentration gradient, and dx is the length of the tube (that is, tube length L). The inner area A of the tube can be calculated using the radius r shown in FIG. 3C.

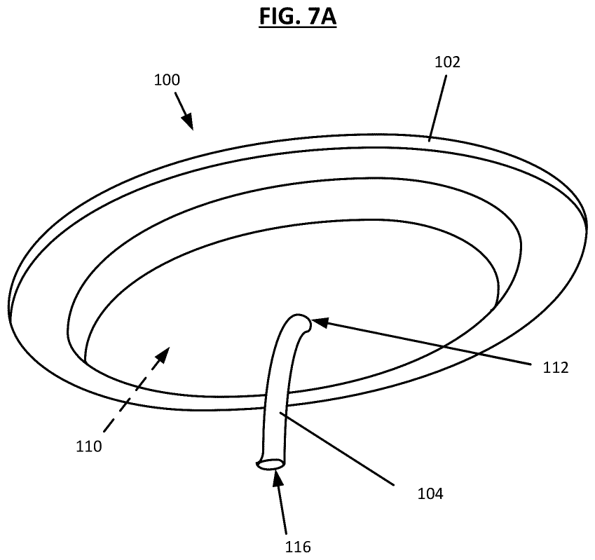

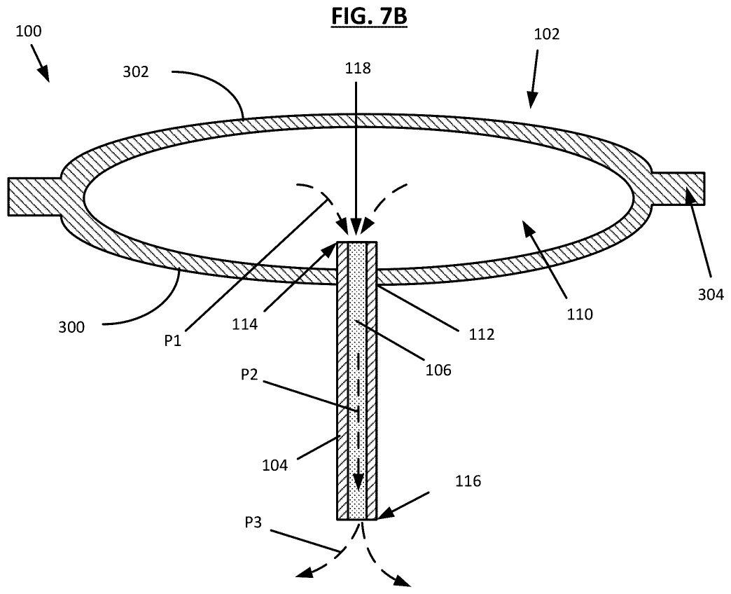

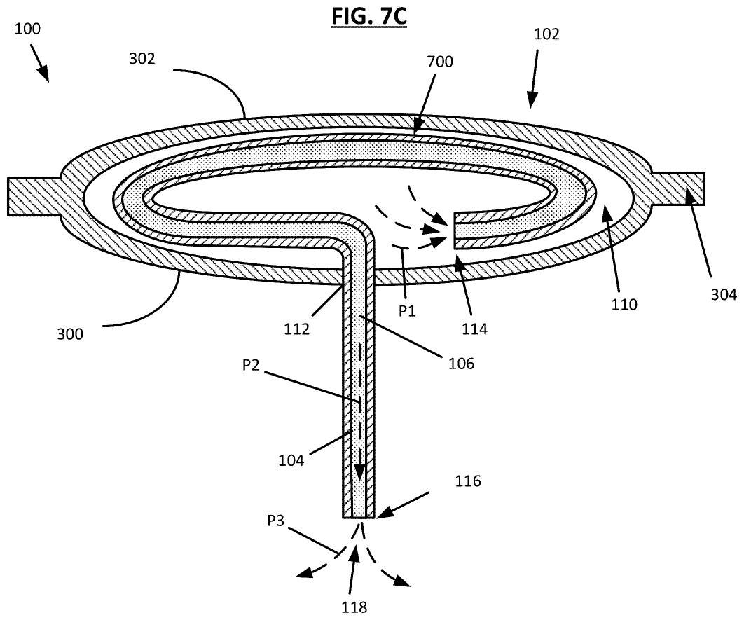

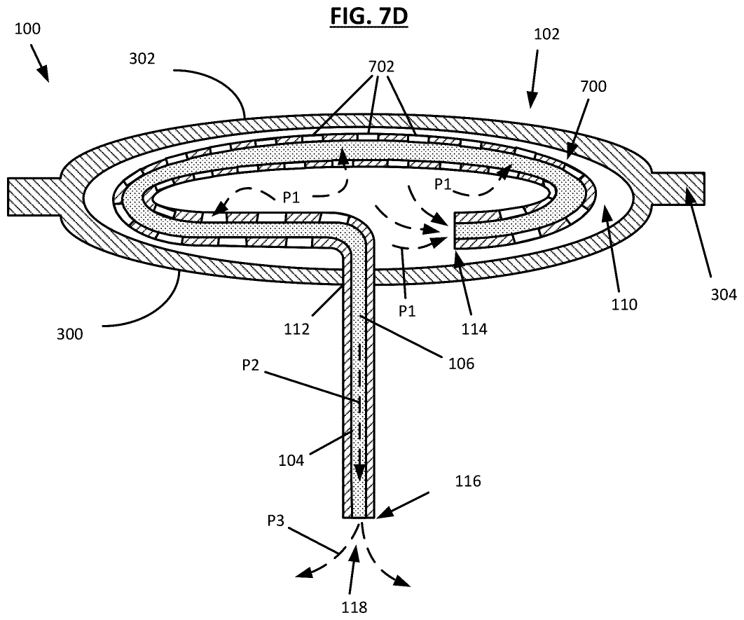

As such, based on the aforementioned Equations, when the porosity of the core 106 is decreased, the amount of material in the core 106 increases, which operates to decrease the effective area of elution out of the tube 104. Furthermore, the length of the tube (dx) is inversely proportional to the rate of medicament transfer, such that increasing the tube length L also facilitates lowering the rate of medicament transfer. Such lengthening can be observed in the example shown in FIG. 7C, which is further explained herein.

Furthermore, the rate is proportional to the diffusivity D, which is affected by tortuosity within the microstructure of the core 106, which can be controlled by altering the microstructure of the core 106 such as the positioning and interconnection among nodes 306 and fibrils 308 that constitute the structure of the core 106.

In FIG. 6A, each broken arrowed line represent one possible passage P2 through the nodes 306 and fibrils 308 of the core 106, according to one example of a microstructure of the core 106. The lines pass through the fibrils 308 but cannot pass through the nodes 306 therefore avoiding them as they pass around the nodes 306. In FIG. 6B, the microstructure is altered such that there are more nodes 306 present in the core 106 as compared to the example in FIG. 6A, thereby forcing the passage P2 to travel around each node 306 in the general direction of the diffusion as provided, causing the rate of transport of the medicament to decrease as compared to FIG. 6A. In FIG. 6C, the microstructure is altered such that there are fewer nodes 306 present in the core 106 as compared to the example in FIG. 6A, thereby allowing the passage P2 to travel in a relatively straight line as compared to FIG. 6A, which increases the rate of transport of the medicament through the microstructure due to the passage P2 requiring less circumventing around the nodes 306. As such, the node porosity may define the rate of medicament transport as well as node density. As the passage P2 traveled becomes more tortuous due to denser positioning of nodes 306, the lower the medicament transport rate would become.

In some examples, the node-to-fibril composition ratio and/or the porosity or density of the microstructure may affect the medicament transport rate. For example, when there are fewer nodes and more fibrils, the transport rate would increase. For example, when the microstructure has a more open configuration (greater porosity, lower density), the transport rate would also increase.

FIGS. 7A through 7D show examples of the medicament delivery device 100 according to embodiments disclosed herein. The device 100 includes the port 112 disposed in one of the first surface 300 or the second surface 302 instead of between these surfaces at the periphery 304 as disclosed in FIG. 3B, for example. The figures show the port 112 disposed in the first surface 300 such that the tube 104 extends from the surface that is proximal to the eye or facing toward the eye after implant, but it should be understood that, in some examples, the port 112 may be disposed in the second surface 302 such that the tube 104 may extend around the body portion 102 before reaching the target tissue of the eye in which the second end 116 is configured to be inserted, as suitable.

In FIG. 7B, a substantial portion (i.e., a majority) of the tube 104 (or the length of the tube) is disposed external to the reservoir 110. As such, greater than 50% and less than about 90%, less than about 80%, less than about 70%, less than about 60%, or any other suitable range or value inclusively therebetween of the tube 104 may be located external to the reservoir in view of FIG. 7B. In FIG. 7C, a substantial portion of the tube 104 is disposed internal to the reservoir 110 (referred to as internal portion 700). A substantial portion may be defined as greater than about 50%, greater than about 60%, greater than about 70%, greater than about 80%, greater than about 90%, or any other suitable range or value inclusively therebetween of the total length L of the tube 104. As such, less than about 40%, less than about 30%, less than about 20%, or any other suitable range or value inclusively therebetween of the tube 104 may be located external to the reservoir in view of FIG. 7C. In some examples, between 10% and 40%, inclusive, of the tube 104 may be located external to the reservoir, i.e., the internal portion 700 is between 60% and 90%, inclusive, of the total length L of the tube 104. Therefore, the tube 104 can be arranged within the reservoir 110 so as to increase the length L of the tube 104 as appropriate to control the rate of medicament transfer through the core 106 which may extend through a portion or an entirety of the lumen 118 of the tube 104, since as explained above with regard to Equation 2, increasing the tube length L facilitates lowering the rate of medicament transfer therein.

In FIG. 7D, the internal portion 700 of the tube 104 has a plurality of density diffusion openings 702 where the core 106 is exposed to the reservoir 110. As such, these openings 702 in the tube 104 form a plurality of locations through which the medicament may enter the core 106 by density diffusion via the first passage P1, in order to provide additional means of controlling the metering of the medicament through the core 106, for example.

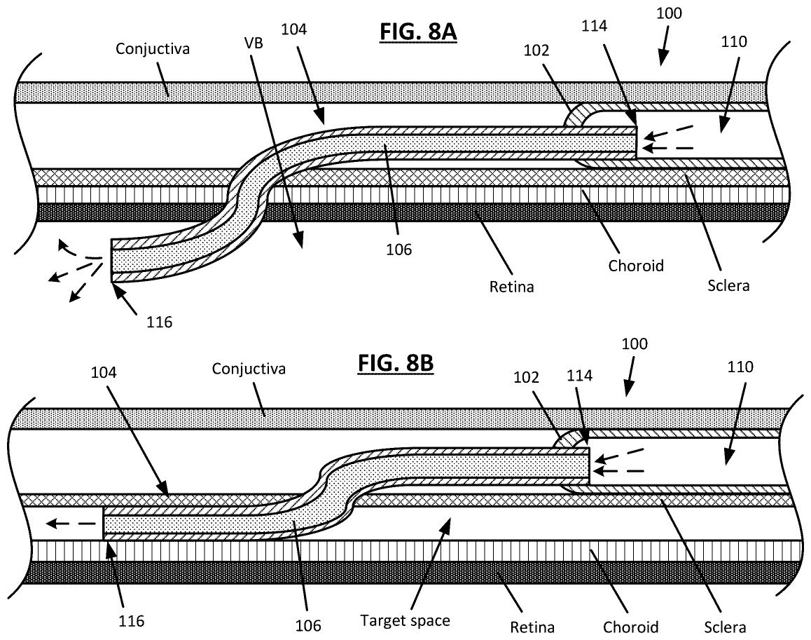

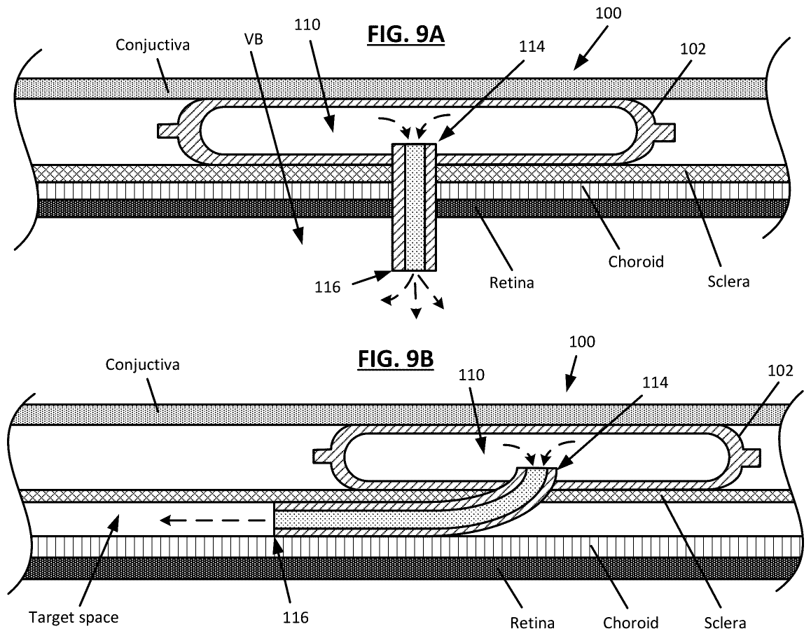

FIGS. 8A and 8B, 9A and 9B, as well as 10A and 10B illustrate how the device 100 may be disposed extraocularly within the different layers of the eye to facilitate intraocular medicament delivery according to embodiments disclosed herein.

In FIGS. 8A and 9A, the body portion 102 is disposed subconjunctivally such that the reservoir 110 rests between the conjunctiva and the sclera, while the tube 104 extends through the sclera, choroid, and retina such that the second end 116 is disposed in the VB to intraocularly deliver the medicament. These examples correspond with the examples illustrated in FIGS. 2C through 2E. In FIGS. 8B and 9B, the tube 104 only extends through the sclera and a portion thereof rests between the sclera and the choroid, allowing the second end 116 to be disposed to facilitate intraocular medicament delivery into a target space defined between the sclera and the choroid. These examples correspond with the example illustrated in FIG. 2F.

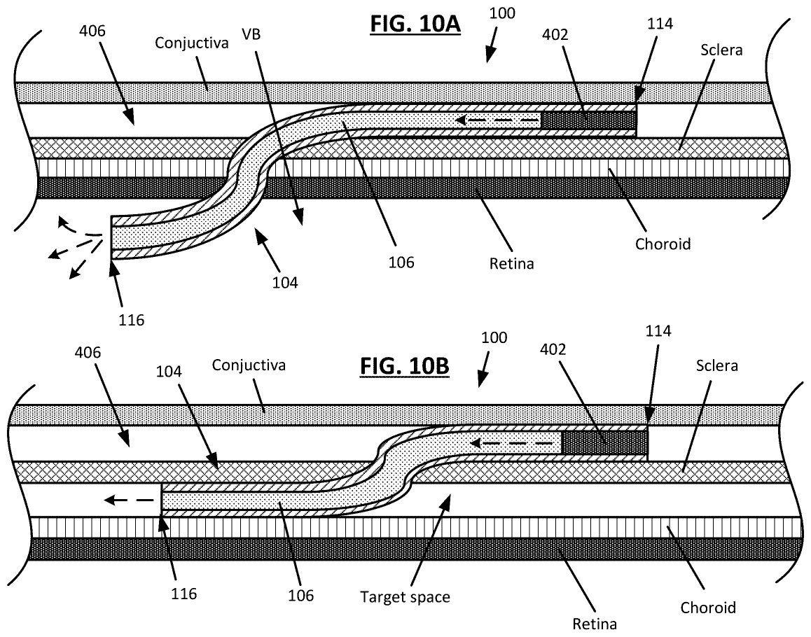

In FIG. 10A, the first end 114 of the tube 104 is disposed subconjunctivally such that the microporous medicament-receiving component 402 rests between the conjunctiva and the sclera, while the tube 104 extends through the sclera, choroid, and retina such that the second end 116 is disposed in the VB to intraocularly deliver the medicament. In FIG. 10B, the tube 104 only extends through the sclera and a portion thereof rests between the sclera and the choroid, allowing the second end 116 to be disposed to facilitate intraocular medicament delivery into a target space defined between the sclera and the choroid.

It should be understood that, although the component 402 is shown for illustrative purposes, any suitable medicament-receiving portion 400 such as the medicament-receiving space 404 may be implemented as well. For example, the medicament may be disposed in the region between the conjunctiva and the sclera, and the first end 114 of the tube 104 may receive the medicament from the region, which would be defined as the surrounding environment 406. It is to be understood that, in such cases, the surrounding environment 406 in which the medicament may be initially and temporarily stored is different from the target space to which the medicament is to be delivered. The target space may be any suitable region within the eye, including but not limited to the VB (FIG. 10A) and the region between the sclera and the choroid (FIG. 10B) as shown.

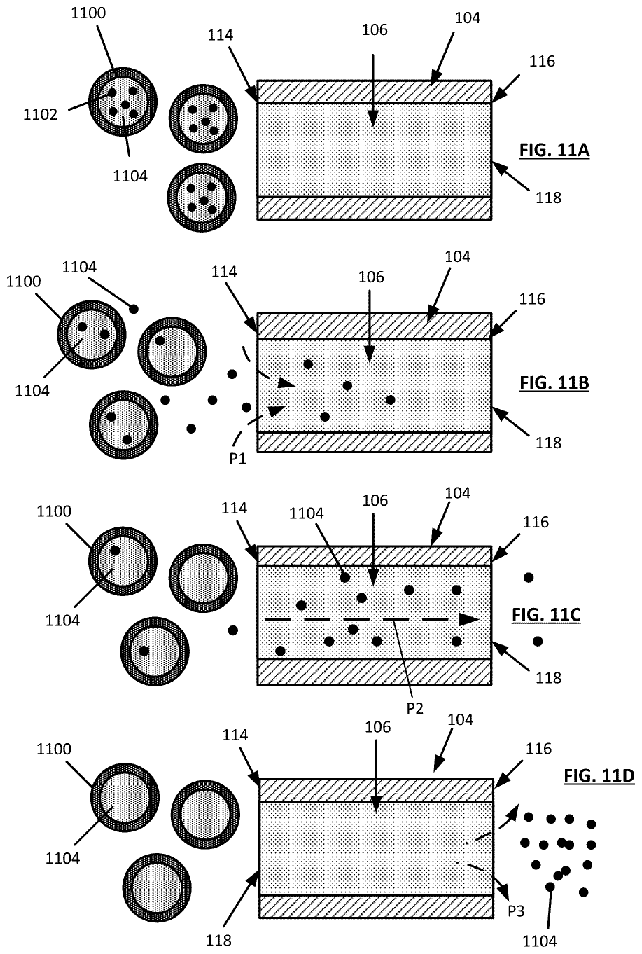

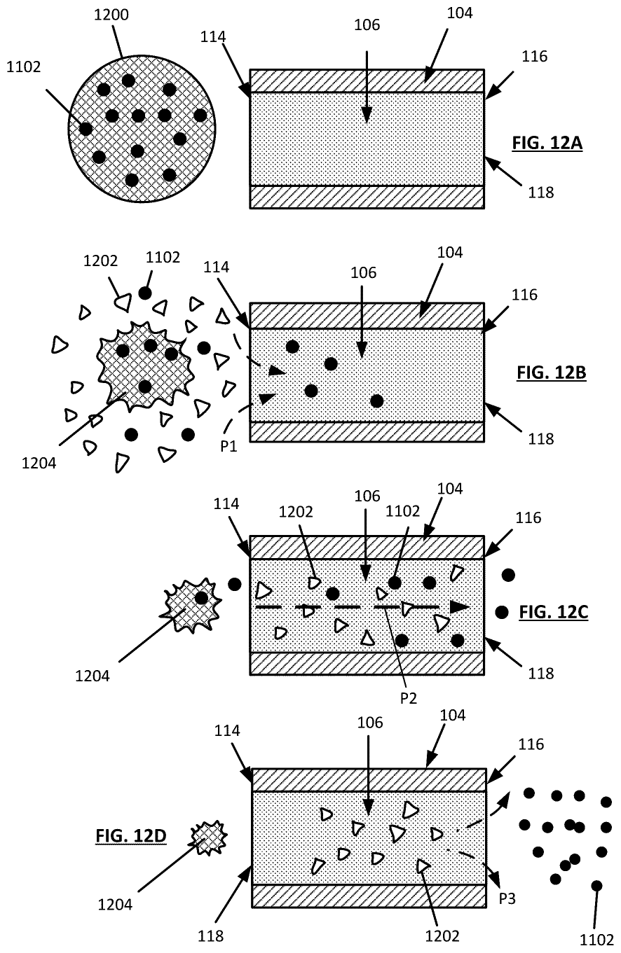

FIGS. 11A through 11D and 12A through 12D illustrate different ways in which the metering of medicament delivery may be achieved using different types of medicament carrier polymers according to embodiments disclosed herein. Any suitable materials for the medicament carrier polymers as well as the delivery process may be implemented as known in the art, such as in Sun T, Zhang Y S, Pang B, Hyun D C, Yang M, Xia Y. “Engineered nanoparticles for drug delivery in cancer therapy.” Angew Chem Int Ed Engl. 2014 Nov 10;53(46):12320-64. doi: 10.1002/anie.201403036. Epub 2014 Oct 7. PMID: 25294565, incorporated herein by reference in its entirety.

In FIG. 11A, nonbiodegradable medicament carrier polymers 1100 (or insoluble polymer membranes) are provided which store therein medicament molecules 1102 that are preloaded for delivery. In some examples, the polymers 1100 may also include aqueous solution 1104 to facilitate controlled delivering the medicament molecules 1102 from the polymers 1100 via diffusion. In some examples, instead of the aqueous solution 1104, the polymers 1100 may be provided with internal polymer matrix to prevent premature release of the medicament molecules 1102, as known in the art.

In FIG. 11B, the polymers 1100 gradually release the medicament molecules 1102 via controlled diffusion, such that the medicament molecules 1102 are released and are no longer restricted within the polymers 1100. In FIG. 11C, the released medicament molecules 1102 travel into the microporous core 106 inside the tube 104 from the first end 114 via passage P1 shown in FIG. 11B, and the medicament molecules 1102 travel from the first end 114 to the second end 116 using diffusion via passage P2. In FIG. 11D, the medicament molecules 1102 are delivered to the target region via passage P3, while the nonbiodegradable medicament carrier polymers 1100 remain on the other side of the tube 104 near the first end 114.

The selective permeance of the medicament molecules 1102 through the core 106 while restricting access of the polymers 1100 is due to the internal microstructure (e.g., the nodes 306 and fibrils 308) of the core 106. Specifically, the internal microstructure is formed so as to preselect the porosity of the core 106 in order to accommodate the molecular shape, molecular mass, and/or molecular polarity of the medicament molecules 1102. For example, the internal microstructure may be preselected to allow a passage of the medicament molecules 1102 that have a predetermined molecular shape, a predetermined molecular mass, and/or a predetermined molecular polarity. That is, for each type of medicament, the microstructure of the core 106 may be altered as suitable so as to prevent any compound or molecule with a larger molecular shape or mass than the selected medicament, or with different molecular polarity from the selected medicament, from entering the core 106 or passing entirely through the core 106 from the first end 114 to the second end 116. Therefore, in some examples, the nodes 306 and fibrils 308 may be capable of capturing the molecules of any larger foreign particles therewithin, which is illustrated in the embodiment of FIGS. 12A through 12D as further explained herein.

In FIG. 12A, the medicament molecules 1102 are preloaded and initially stored in erodible or biodegradable medicament carrier polymers 1200. The polymers 1200 may be tailored to control the erosion kinetics, thereby causing the polymers 1200 to degrade at a predetermined rate to release the medicament molecules 1102. In FIG. 12B, the polymers 1200 gradually erode or degrade into smaller degraded polymer particles 1202 while leaving behind the undegraded polymers 1204.

Once released, as shown in FIG. 12C, the medicament molecules 1102 enter the core 106 from the first end 114 via passage P1, and travel toward the second end 116 via passage P2. In this process, some of the degraded polymer particles 1202 may also enter the microstructure of the core 106, while the undegraded polymers 1204 are left behind outside core 106 near the first end 114 until all of the undegraded polymers 1204 eventually erode into the smaller particles 1202, by which point the particles 1202 may enter the core 106.

In FIG. 12D, the medicament molecules 1102 are delivered to the target region from the second end 116 via passage P3. In some examples, the microstructure of the core 106 captures the polymer particles 1202 therein so as to prevent the particles 1202 from entering the target region. This may be beneficial to reduce the amount of carrier polymers from entering the target region or to prevent the carrier polymers from entering the target region until they are eroded to become small enough to pass through the microstructure. Either way, the microstructure of the core 106 can reduce any large foreign particles from being delivered to the target region, thereby increasing safety of the device 100 for its users.

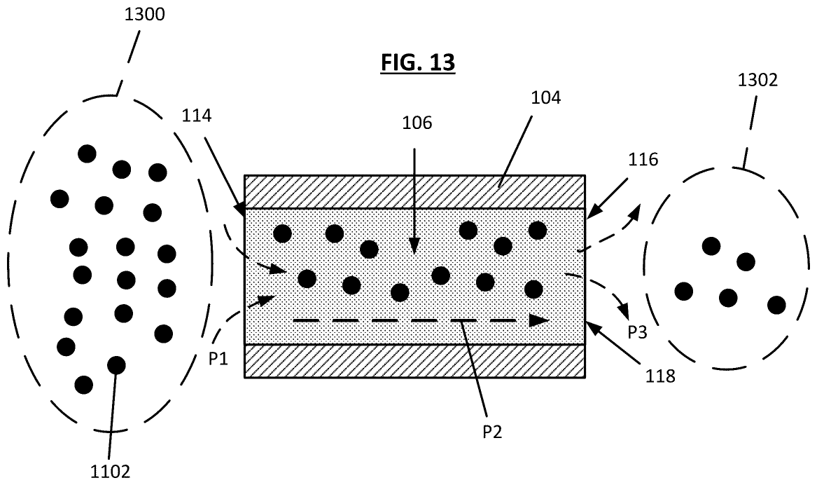

In some examples, as shown in FIG. 13, the medicament molecules 1102 may be delivered from the first end 114 to the second end 116 according to a difference in concentration (concentration gradient) of the medicament molecules 1102 between the two ends 114, 116. Concentration gradient is widely used for transporting fluid and medicaments in the medical field, as explained in Sulaiman, D & Suhaimi, H & Shamsuddin, Norazanita. (2020). “Estimating glucose diffusion coefficient of membranes for tissue engineering applications using Fick's First Law.” IOP Conference Series: Materials Science and Engineering. 991. 012103. 10.1088/1757-899X/991/1/012103, which is incorporated herein by reference in its entirety. As such, the concentration gradient indicating that the concentration of medicament molecules 1102 is greater at the first end 114 (or a higher-concentration region 1300 surrounding the first end 114) than the second end 116 (or a lower-concentration region 1302 surrounding the second end 116) causes the medicament molecules 1102 to be transported by diffusion via passage P2 through the core 106. The medicament transport thus continues until there is no longer a concentration gradient, i.e. when the concentrations of the medicament molecules 1102 in the regions 1300 and 1302 are the same.

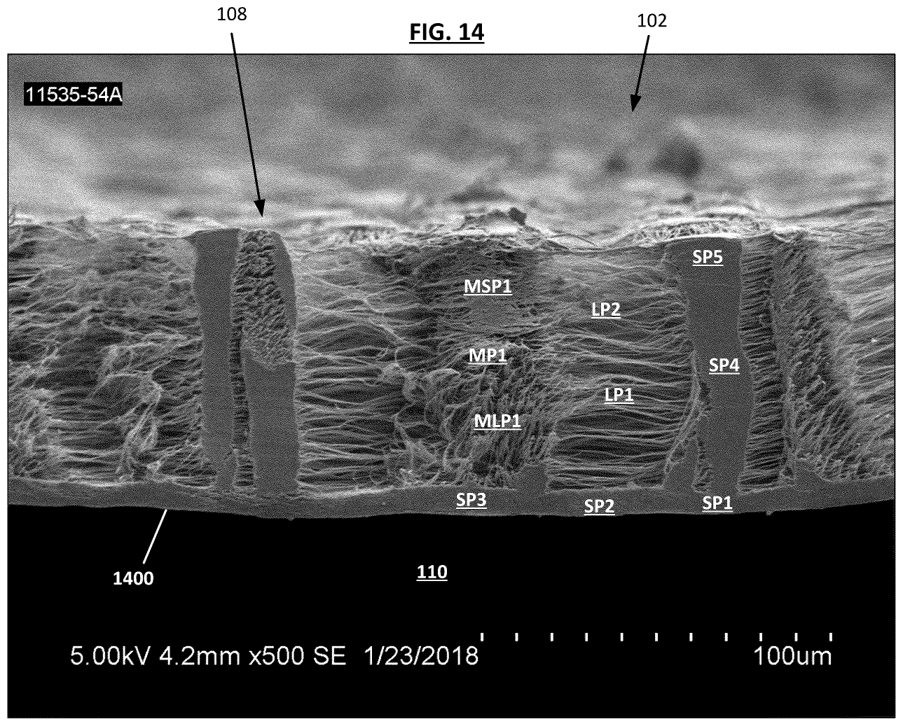

FIG. 14 shows a microscopic view of a microporous material of the external surface 108 of the body portion 102 of the medicament delivery device 100 according to some embodiments. Displayed at the bottom of FIG. 14 is: “5.00 kV 4.2 mm×500 SE Jan. 23, 2018,” and the distance between two consecutive lines as shown at the bottom right hand corner represents 10 μm. For example, the microporous material of FIG. 14 may be referred to throughout with reference to a medical implant device or system. As can be appreciated by a person of skill in the art and with reference to FIG. 14, the microporous aspects and parameters of the microporous material can be defined in a variety of ways. In an application of a microporous material in an ocular device, such as the medicament delivery device 100 described herein, configured for in situ placement in the tissue of the eye to facilitate the delivery of a medicament to the eye for treatment of a disease, the microporous properties of such a microporous material can be generally characterized by a volumetric porosity value that can be defined as a ratio of a volume of the air or fluid defined by and contained within the microporous material as compared to an overall volume (or total volume) of the microporous material.

In another definition, a volumetric porosity can be defined as a percentage of the microporous material volume that is occupied by non-structural or transient elements such as air or other fluids. For example, a microporous material with an overall volume of 100 mm³ and with 30 mm³ of that volume comprising chambers holding air or a fluid would have a volumetric porosity value of 0.3 because 30% of the volume of the microporous material is empty or transient space that is filled with air or other fluids.

As can be appreciated, two microporous materials can have the same volumetric porosity but differ in the pore sizes presented to the incoming or exiting air or fluid. For example, a first material can a have a small number of large pores distributed over a fixed overall volume and a second material can have a relatively large number of relatively smaller pores distributed over the same fixed volume, and both microporous materials could have the same volumetric porosity if the air/fluid volume of the two materials are the same.

As can be further appreciated, the properties of the microporous materials used in the device can also be defined by the size of the passages passing through the microporous material or similarly defined as a pore size measured where a passage terminates at a surface of the microporous material or measured along a length of a passage within the material. Microporous materials with small pores or passages can impede flow through the material and comparatively large pores or passages can provide an increased pass through of the air or fluid into, out of, or within the microporous material.

As can be still further appreciated, the properties of the microporous material can also be defined by a tortuosity of the passages entering into and passing through the material, with relatively small or large passages presenting impeded fluid pathways due the frequency of turns in the passages or by the placement of obstructions in the fluid pathways. The air/fluid passthrough rates of a microporous material can be managed by controlling or defining any of the above-described characteristics of the material to provide a suitable material for use to facilitate delivery of a medicament to the eye for the treatment of a disease.

For simplicity, the aforementioned characteristics and variables of the microporous material used in the various embodiments and examples described herein can be presented simply as a porosity which can be based on a volumetric porosity, a pore or passage size, or a tortuosity metric. Again, with reference to FIG. 14, internal portions of the microporous material can have varying porosities (or volumetric porosities, or pore sizes, or tortuosities). The internal portions can extend between an inner surface 1400 and the external surface 108.

At any of these portions of a body portion 102, the porosity can comparatively range in degree from small pore size (SP), medium-small pore size (MSP), medium pore size (MP), medium-large pore size (MLP), and large pore size (LP). Assuming, for discussion purposes here, that delivery travels along a relatively straight path through a microporous material so as to sequentially engage porosities of the inner surface 1400, a uniform internal portion, and the external surface 108, the combined flow resistance can be represented by likewise concatenating their respective porosities. For instance, the inner surface 1400 typically has a low porosity throughout (e.g., to resist tissue ingrowth into the reservoir 110), and portions of the interior portions and the external surface 108 can have any of the aforementioned degrees of porosity. Under these circumstances when the internal portion has a medium porosity and, for example, the internal portions have a medium porosity and the external surface 108 has a high porosity, the medicament delivery through the microporous material from the reservoir 110 to tissue surrounding the device can be represented as SP-MP-LP. More examples are discussed here below.

Various delivery paths can be present within the microporous material. Relatively linear flow paths may comprise regions SP1-SP4-SP5, for example or SP3-MLP1-MP1-MSP1. Although some flow paths may be relatively straight, there are also flow paths that are nonlinear. For instance, under certain conditions, at least some flow may proceed to flow through areas of increasingly less resistance such as SP1-LP1-LP2 or SP3-MLP1-LP1-LP2. As will be appreciated, the microstructure of the microporous materials may undergo modification processes to obtain certain types of flow through the microstructure. For instance, the microstructure may have relatively uniform layers across layered within the microstructure, or as shown here, have variable portions throughout the thickness of the microporous material.

In some examples, the body portion 102 defines a wall portion thickness extending between the inner surface 1400 and the external surface 108. The wall portion thickness can define an internal region of the body portion 102 having a transition porosity that is between a porosity of the low porosity surface (e.g., having smaller pore sizes) of the inner surface 1400 and a porosity of the high porosity surface (e.g., having larger pore sizes) of the external surface 108. In addition, or in alternative, the internal region can have an internal region porosity that is equal to porosities of the low porosity surfaces of the inner surface 1400 and the external surface 108. In addition, or in alternative, the internal region can have an internal region porosity that is equal to a porosity of the low porosity surface of the inner surface 1400. In addition, or in alternative, the internal region can have an internal region porosity that is equal to a porosity of the high porosity surface of the external surface 108.

With reference still to the microporous material shown in FIG. 14, the fluid pathways may also be impacted by the concentration gradient between a fluid such as water and the medicament that is within the reservoir 110. More specifically, during the process of medicament delivery, the medicament is first contained within the reservoir 110. Fluid may then be delivered through the microporous material and into the reservoir 110, causing the medicament to leach out of the microporous material and to the targeted delivery site. While described herein as having layers or stratum, the microporous material may lack separate and distinct layers but instead comprise different areas of varying porosity for the fluid and medicament to travel though, as previously described. The medicament delivery device 100, and more particularly the microporous material of the medicament delivery device 100, may also be optimized for targeted delivery. In other words, the areas at which the microporous material is incorporated may be chosen in order to allow for medicament delivery only in the target location around the device 100.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

What is claimed is:

1. A medicament delivery device for metering delivery of a medicament over a time period to an ocular tissue, the device comprising:
a body portion having an external surface defining an internal reservoir, the external surface defining a port of the body portion disposed to facilitate a first passage of the medicament from the reservoir;
a tube having a first end, a second end opposing the first end, and a lumen extending therebetween along a tube length, the tube length defining a second passage of the medicament from the first end to the second end, the first end coupled to the port to receive the first passage of the medicament from the reservoir, the second end disposed at a distance from the first end to deliver the medicament to the ocular tissue; and
a microporous core disposed in the lumen along at least a portion of the tube length, the microporous core having a porosity that meters the second passage of the medicament.

2. The device of claim 1, wherein the medicament has a first state when disposed at the first end of the tube and a second state when disposed at the second end of the tube, the medicament transitioning from the first state to the second state when travelling along the second passage through the microporous core.

3. The device of claim 2, wherein the first state being a delivery state of the medicament and the second state being a therapeutic state of the medicament.

4. The device of claim 2, wherein the first state being a non-therapeutic state of the medicament and the second state being a therapeutic state of the medicament.

5. The device of claim 2, wherein the first state being a high-concentration state of the medicament and the second state being a low-concentration state of the medicament, the low-concentration state having a lower concentration than the high-concentration state.

6. The device of claim 1, wherein the microporous core includes a plurality of nodes and fibrils configured to meter the second passage of the medicament.

7. The device of claim 6, wherein the second passage of the medicament is configured to be metered by a surface roughness and a surface energy of the plurality of nodes and fibrils of the microporous core.

8. The device of claim 1, wherein the tube is a coating disposed on an external surface of the microporous core.

9. The device of claim 1, wherein the tube is a tape wrapped around an external surface of the microporous core.

10. The device of claim 1, wherein the porosity of the microporous core is preselected to allow a passage of the medicament compound having a predetermined molecular shape.

11. The device of claim 1, wherein the porosity of the microporous core is preselected to allow a passage of the medicament compound having a predetermined molecular mass.

12. The device of claim 1, wherein the porosity of the microporous core is preselected to allow a passage of the medicament compound having a predetermined molecular polarity.

13. The device of claim 1, wherein the external surface of the body portion comprises a first surface and a second surface opposing the first surface, and the port is disposed between the first and second surfaces.

14. The device of claim 1, wherein the external surface of the body portion comprises a first surface and a second surface opposing the first surface, and the port is disposed in the first surface.

15. The device of claim 14, wherein the first end of the tube is disposed within the reservoir such that a majority of the tube length is disposed within the reservoir.

16. The device of claim 15, wherein the majority of the tube that is disposed within the reservoir is configured to partially cover the microporous core to provide a plurality of density diffusion openings.

17. The device of claim 15, wherein from 10% to 40% of the tube length is located external to the reservoir.

18. The device of claim 1, wherein the body portion includes a resealable opening through which the reservoir is configured to be refilled with the medicament to be delivered.

19. A medicament metering device for metering delivery of a medicament over a time period to an ocular tissue, the medicament metering device comprising:
a tube having a first end, a second end opposing the first end, and a lumen extending therebetween along a tube length; and
a microporous core disposed in the lumen along at least a portion of the tube length, the microporous core having a porosity that facilitates a metered passage of the medicament through the microporous core from the first end of the tube towards the second end of the tube
wherein the medicament has a first state when disposed at the first end of the tube and a second state when disposed at the second end of the tube, the medicament transitioning from the first state to the second state when travelling along the metered passage, and
wherein the first end of the tube includes a medicament-receiving portion sized to receive the medicament in the first state and positioned to channel the medicament in the first state to the microporous core.

20. The device of claim 19, wherein the first state being a delivery state of the medicament and the second state being a therapeutic state of the medicament.

21. The device of claim 19, wherein the first state being a non-therapeutic state of the medicament and the second state being a therapeutic state of the medicament.

22. The device of claim 19, wherein the first state being a high-concentration state of the medicament and the second state being a low-concentration state of the medicament, the low-concentration state having a lower concentration than the high-concentration state.

23. The device of claim 19, wherein the medicament-receiving portion comprises a microporous medicament-receiving component having a different porosity from the porosity of the microporous core.

24. The device of claim 19, wherein the first end is substantially closed and the medicament-receiving portion is a medicament-receiving space within the lumen in which the medicament is temporarily stored for the delivery.

25. The device of claim 19, wherein the first end is substantially open and the medicament-receiving portion is configured to receive the medicament from a surrounding environment external to the tube.

26. The device of claim 25, further comprising a filtering membrane disposed at the first end and configured to filter the medicament received from the surrounding environment.

27. The device of claim 25, wherein the first end is angulated with respect to the tube length.

28. The device of claim 25, wherein a section of the microporous core proximate to the first end of the tube is angulated with respect to the tube length.

29. The device of claim 27, wherein a section of the microporous core proximate to the first end of the tube is angulated with respect to the tube length.

30. The device of claim 25, further comprising:
a body portion having an external surface defining an internal reservoir, the external surface defining a port of the body portion coupled with the first end of the tube and disposed to deliver the medicament from the reservoir to the tube, wherein the surrounding environment is the reservoir of the body portion.

31. The device of claim 19, wherein the microporous core includes a plurality of nodes and fibrils configured to control the metered passage of the medicament.

32. The device of claim 31, wherein the second passage of the medicament is configured to be metered by a surface roughness and a surface energy of the plurality of nodes and fibrils of the microporous core.

33. The device of claim 19, wherein the tube is a coating disposed on an external surface of the microporous core.

34. The device of claim 19, wherein the tube is a tape wrapped around an external surface of the microporous core.

35. The device of claim 19, wherein the porosity of the microporous core is preselected to allow a passage of the medicament compound having a predetermined molecular shape.

36. The device of claim 19, wherein the porosity of the microporous core is preselected to allow a passage of the medicament compound having a predetermined molecular mass.

37. The device of claim 19, wherein the porosity of the microporous core is preselected to allow a passage of the medicament compound having a predetermined molecular polarity.
